(19) United States
(12) Patent Application Publication
(10) Pub. No.: US 2008/0287663 A1
(43) Pub. Date: Nov. 20, 2008

(54) Process For Production Of 4-Biphenylyazetidin-2-Ones

(75) Inventors: Peter Lee, Herndon, VA (US); Stephen Antonelli, Lynn, MA (US); Regina Lundrigan, Charlestown, MA (US); Eduardo J. Martinez, St. Louis, MO (US); Wayne C. Schairer, Westboro, MA (US); John J. Talley, Somerville, MA (US)

Correspondence Address: HESLIN ROTHENBERG FARLEY & MESITI PC, 5 COLUMBIA CIRCLE, ALBANY, NY, 12203, US

(73) Assignee: MICROBIA, INC., Cambridge, MA (US)

(21) Appl. No.: 11/913,587

(22) PCT Filed: May 8, 2006
(86) PCT No.: PCT/US06/17706

§ 371 Date: Feb. 4, 2008

Related U.S. Application Data
(60) Provisional application No. 60/678,336, filed on May 6, 2005.
(60) Provisional application No. 60/726,929, filed on Oct. 14, 2005.

(51) Int. Cl. C07H 7/04

(52) U.S. Cl. 536/17.4

(57) ABSTRACT

The present invention relates to processes for the production of 4-biphenylylazetidin-2-one derivatives of formula

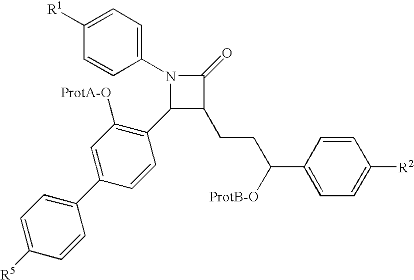

### FIELD OF THE INVENTION

The present invention relates to processes for the production of 4-biphenylylazetidinone derivatives.

### BACKGROUND OF THE INVENTION

(1S)-1,5-Anhydro-1-(4′-{(2S,3R)-3-[(3S)-3-(4-fluorophenyl)-3-hydroxypropyl]-4-oxo-1-phenylazetidin-2-yl}-3′-hydroxybiphenyl-4-yl)-D-glucitol (ADG)

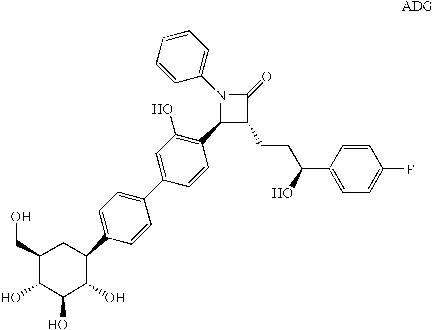

has been shown to be an inhibitor of cholesterol absorption. (See copending U.S. application Ser. No. 10/986,570, which is incorporated herein by reference.)

ADG is a member of the family of azetidinone cholesterol absorption inhibitors. 1,4-Diphenylazetidin-2-ones and their utility for treating disorders of lipid metabolism are described in U.S. Pat. No. 6,498,156 and PCT application WO02/50027, the disclosures of which are incorporated herein by reference. Perhaps the most well-known member of the class of 1,4-diphenylazetidin-2-one hypocholesterolemics is ezetimibe, which is sold as ZETIA™.

U.S. Pat. Nos. 5,631,365; 6,093,812; 5,306,817 and 6,627,757, for example, disclose and claim processes for the preparation of azetidinone derivatives related to ezetimibe.

The present invention is directed toward a process for preparation of ADG and similar saccharide-substituted 4-(biphenylyl)azetidin-2-ones.

### SUMMARY OF THE INVENTION

The present invention relates to processes for preparing ADG-related compounds of the formula Ia

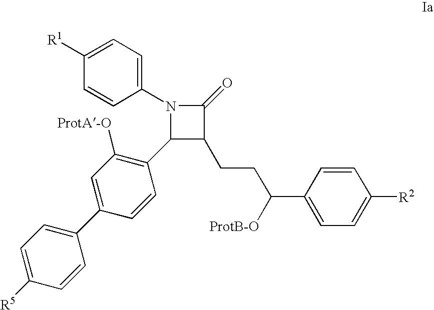

wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁵ is a sugar or a protected sugar.

In a first process aspect, the invention relates to a process comprising reacting a compound of formula IIb

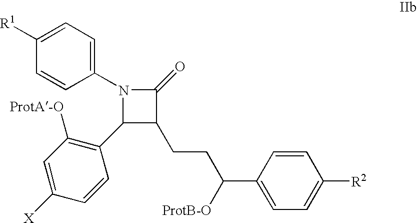

wherein X is chosen from iodine, bromine, chlorine, toluenesulfonyl, methanesulfonyl and trifluoromethanesulfonyl, with a compound of formula III

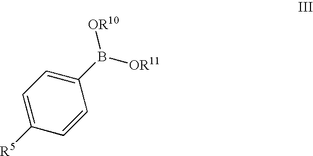

wherein R¹⁰ and R¹¹ are independently selected from H and (C₁-C₆) alkyl, or R¹⁰ and R¹¹ together form a 5-6 membered ring.

Inversely, one may react a compound of formula IIa

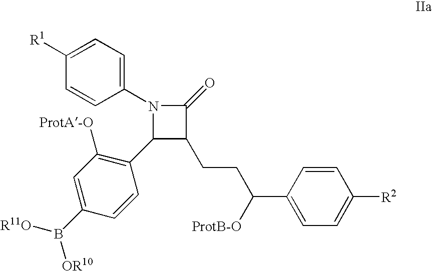

with a compound of formula XX

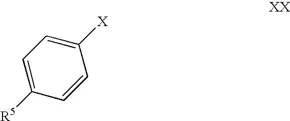

In a second process aspect, the invention relates to a process for preparing a compound of structure II

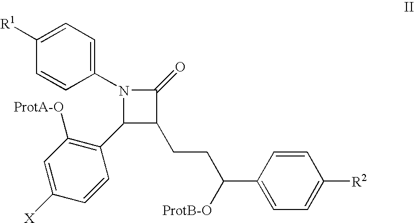

in which ProtA-O— is a protecting group for a phenol chosen from an oxymethyl ether, an allyl ether, a tertiary alkyl ether, a benzyl ether and a silyl ether. The process comprises cyclizing a compound of formula IVa

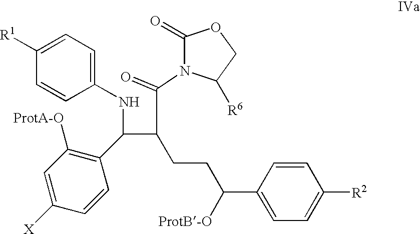

wherein R⁶ is phenyl or benzyl and ProtB′-O— is a protecting group for a benzylic alcohol chosen from an oxymethyl ether, a tetrahydropyranyl or tetrahydrofaranyl ether, methoxycyclohexyl ether, a methoxybenzyl ether, a silyl ether and an ester.

In a third process aspect, the invention relates to a process for preparing a compound of structure IVi

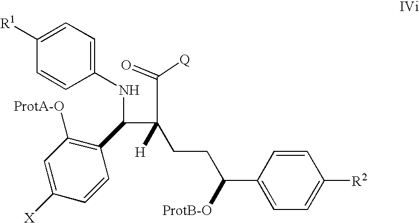

wherein Q is a chiral auxiliary. The chiral auxiliary is chosen from single enantiomers of triphenyl glycol and cyclic and branched nitrogen-containing moieties possessing at least one chiral center. The process comprises reacting a compound of formula V

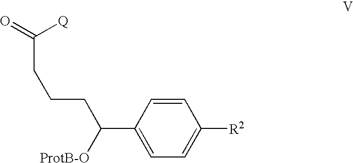

with a compound of formula VI

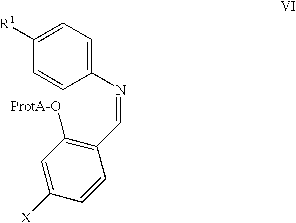

In a fourth process aspect, the invention relates to a process for preparing an imine of formula VI

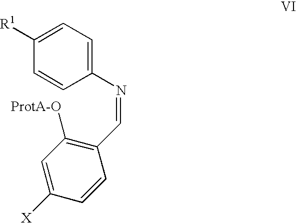

The process comprises (1) reacting a phenol of formula

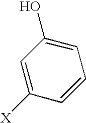

with a source of formaldehyde, followed by (2) Schiff base formation by reacting with an aniline of formula

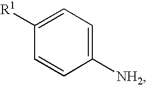

followed by (3) protecting with ProtA.

In a further process aspect, the invention relates to a process for preparing a compound of formula XII:

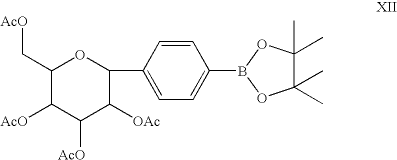

comprising:
(1) treating a protected sugar lactone of formula

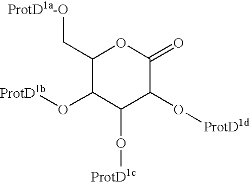

wherein ProtD^{1a}, ProtD^{1b}, ProtD^{1c} and ProtD^{1d} are trialkylsilyl groups, with a Grignard reagent of formula

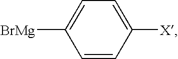

wherein X′ is Br or Cl, followed by methanol and an acid to provide a compound of formula XIII:

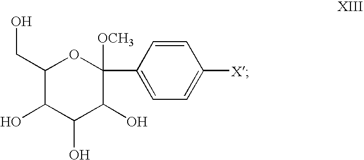

(2) treating XIII with an excess of an acetylating reagent chosen from acetic anhydride, acetyl chloride, and pentafluorophenyl acetate in the presence of a base and acetylimidazole in the presence of a platinum catalyst to provide XIV:

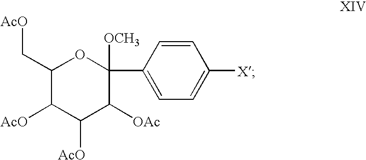

(3) reducing XIV with a silane and a Lewis acid to provide XV:

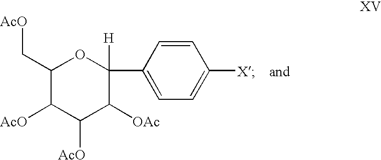

(4) reacting XV with bis(pinacolato)diboron in the presence of a palladium catalyst to produce XII:

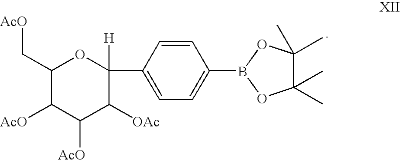

In combination, the processes of the invention provide an overall process for preparing ADG:

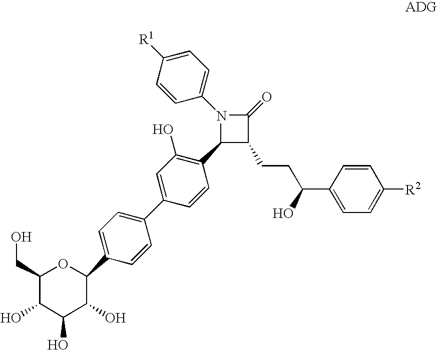

(in which R¹ is H and R² is F) from

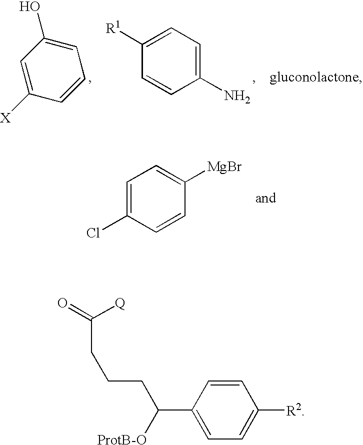

In a product aspect, the invention relates to compounds of formula VI.

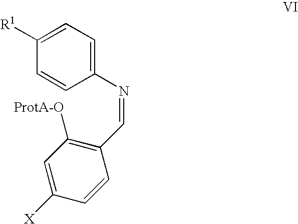

When R¹ is H, X is Br and ProtA is benzyl, the compound must be in solid form and greater than 95% pure.

In a second product aspect, the invention relates to compounds of formula

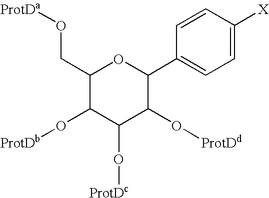

wherein X is chosen from iodine, bromine, chlorine, toluenesulfonyl, methanesulfonyl and trifluoromethanesulfonyl; and ProtD^{a}, ProtD^{b}, ProtD^{c} and ProtD^{d} are protecting groups for a sugar chosen independently from benzyl, silyl (e.g. tBDMS and TMS), acyl (e.g. acetyl and benzoyl), ketal (e.g. acetonide and MOM), and acetal (e.g. benzylidene).

In a third product aspect, the invention relates to compounds of formula:

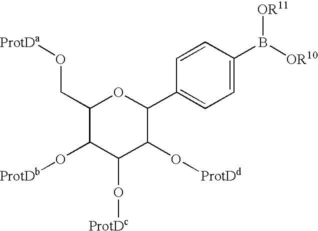

wherein ProtD^{a}, ProtD^{b}, ProtD^{c} and ProtD^{d} are protecting groups for a sugar chosen independently from benzyl, silyl (e.g. TBDMS and TMS), acyl (e.g. acetyl and benzoyl), ketal (e.g. acetonide and MOM), and acetal (e.g. benzylidene); and R¹⁰ and R¹¹ are independently selected from H and (C₁-C₆) alkyl, or R¹⁰ and R¹¹ together form a 5-6 membered ring. In one embodiment, R¹⁰ and R¹¹ together form a dioxaborole:

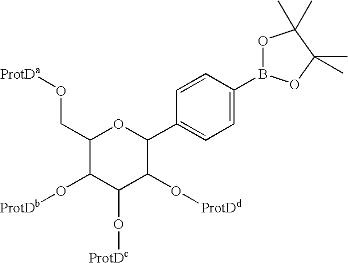

### DETAILED DESCRIPTION OF THE INVENTION

Throughout this application, various references are cited. The disclosures of each of these publications in their entireties are hereby incorporated by reference as if written herein.

### DEFINITIONS

In this specification the terms and substituents are defined when introduced and retain their definitions throughout. The structural depictions of species and genera of the invention are numbered to assist the reader. In general, compounds that share a common core share a common Roman numeral designation. The Roman numeral without further extension generally represents the “parent” genus in its fall breadth; a letter extension indicates a subgenus in which at least one substituent has a more limited range; an italicized i indicates a subgenus or species having a more limited chirality than its parent genus, subgenus or species.

Alkyl is intended to include linear, branched, or cyclic hydrocarbon structures and combinations thereof. When not otherwise restricted, the term refers to alkyl of 20 or fewer carbons. Lower alkyl refers to alkyl groups of 1, 2, 3, 4, 5 and 6 carbon atoms. Examples of lower alkyl groups include methyl, ethyl, propyl, isopropyl, butyl, s-and t-butyl and the like. Preferred alkyl and alkylene groups are those of C₂₀ or below (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀). Cycloalkyl is a subset of alkyl and includes cyclic hydrocarbon groups of 3, 4, 5, 6, 7, and 8 carbon atoms. Examples of cycloalkyl groups include c-propyl, c-butyl, c-pentyl, norbornyl, adamantyl and the like.

C₁ to C₂₀ Hydrocarbon (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀) includes alkyl, cycloalkyl, alkenyl, alkynyl, aryl and combinations thereof. Examples include benzyl, phenethyl, cyclohexylmethyl, camphoryl and naphthylethyl. The term “phenylene” refers to ortho, meta or para residues of the formulae:

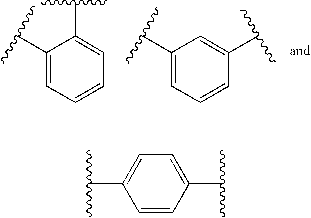

Alkoxy or alkoxyl refers to groups of 1, 2, 3, 4, 5, 6, 7 or 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to six carbons.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Polyol refers to a compound or residue having a plurality of —OH groups. Polyols may be thought of as alkyls in which a plurality of C—H bonds have been replaced by C—OH bonds. Common polyol compounds include for example glycerol, erythritol, sorbitol, xylitol, mannitol and inositol. Linear polyol residues will generally be of the empirical formula —C_{y}H_{2y+1}O_{y}, and cyclic polyol residues will generally be of the formula —C_{y}H_{2y-1}O_{y}. Those in which y is 3, 4, 5 and 6 are preferred. Cyclic polyols also include reduced sugars, such as glucitol.

Acyl refers to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, propionyl, isobutyryl, t-butoxycarbonyl, benzoyl, benzyloxycarbonyl and the like. Lower-acyl refers to groups containing one to six carbons.

Aryl and heteroaryl refer to aromatic or heteroaromatic rings, respectively, as substituents. Heteroaryl contains one, two or three heteroatoms selected from O, N, or S. Both refer to monocyclic 5- or 6-membered aromatic or heteroaromatic rings, bicyclic 9- or 10-membered aromatic or heteroaromatic rings and tricyclic 13- or 14-membered aromatic or heteroaromatic rings. Aromatic 6, 7, 8, 9, 10, 11, 12, 13 and 14-membered carbocyclic rings include, e.g., benzene, naphthalene, indane, tetralin, and fluorene and the 5, 6, 7, 8, 9 and 10-membered aromatic heterocyclic rings include, e.g., imidazole, pyridine, indole, thiophene, benzopyranone, thiazole, faran, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine.

The term “sugar” is used in its normal sense, as defined in Hawley's Condensed Chemical Dictionary, 12^{th} Edition, Richard J. Lewis, Sr.; Van Nostrand Reinhold Co. New York. It encompasses any carbohydrate comprised of one or two saccharose groups. The monosaccharide sugars (often called simple sugars) are composed of chains of 2-7 carbon atoms. One of the carbons carries aldehydic or ketonic oxygen, which may be combined in acetal or ketal forms. The remaining carbons usually have hydrogen atoms and hydroxyl groups (or protecting groups for hydroxyl, such as acetate). Among monosaccharides which would be considered within the term “sugars” as intended in this application, are arabinose, ribose, xylose, ribulose, xylulose, deoxyribose, galactose, glucose, mannose, fructose, sorbose, tagatose, facose, quinovose, rharnnose, manno-heptulose and sedoheptulose. Among the disaccharides are sucrose, lactose, maltose, and cellobiose. Unless specifically modified, the general term “sugar” refers to both D-sugars and L-sugars. The sugar may also be protected. The sugar may be attached through oxygen (as in U.S. Pat. No. 5,756,470) or through carbon (as in PCT WO 2002066464), the disclosures of both of which are incorporated herein by reference.

Reduced C-attached sugars or C-glycosyl compounds are also encompassed by the invention. The reduced sugars (e.g. glucitol), which could be classed either as polyols or as sugars, are also known as alditols. Alditols are polyols having the general formula HOCH₂[CH(OH)]ₙCH₂OH (formally derivable from an aldose by reduction of the carbonyl group).

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group that is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry [See e.g. Protective Groups in Organic Synthesis by T. W. Greene and P. G. M. Wuts, 2nd Edition; John Wiley & Sons, New York (1991)].

In processes of the invention, one may contemplate, for example, for the protection of the hydroxyls on the sugar, acetic anhydride, acetyl chloride or pentafluorophenyl acetate in the presence of a base and acetylimidazole in the presence of a platinum catalyst. The acetyl may be cleaved at the appropriate stage with base (e.g. potassium carbonate in aqueous methanol, guanidine in ethanol, lithium hydroxide in aqueous methanol, triethylamine in methanol, methanolic ammonia), with potassium cyanide in ethanol or with a source of fluoride ion (e.g. potassium fluoride or cesium fluoride) in methanol. For protection of the non-sugar alcohols, (e.g. ProtA and ProtB) one may contemplate, for example, benzyl ethers. The benzyl may be unsubstituted or substituted (e.g. p-methoxybenzyl, dimethoxybenzyl, trimethoxybenzyl, nitrobenzyl, halobenzyl, and the like).

The abbreviations Me, Et, Ph, Tf, Ts and Ms represent methyl, ethyl, phenyl, trifluoromethanesulfonyl, toluensulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. As understood by one skilled in the art, the terms “isopropanol”, “isopropyl alcohol” and “2-propanol” are equivalent and represented by CAS Registry No: 67-63-0.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. Thus, the formula XI is intended to encompass both of the pure enantiomers of that pair:

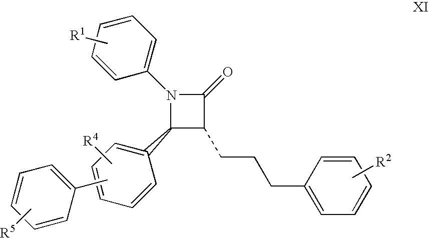

Means either pure 3R,4S:

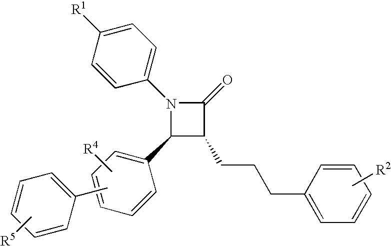

or pure 3S,4R:

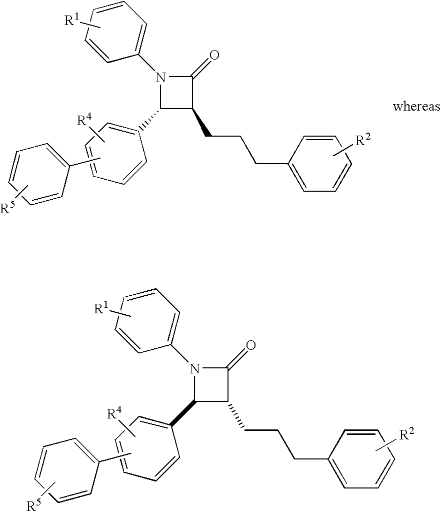

refers to a racemic mixture of R,S and S,R, i.e. having a trans relative configuration on the beta lactam ring.

The term “enantiomeric excess” is well known in the art and is defined for a resolution of ab into a+b as

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

ADG-related compounds of the formula Ia

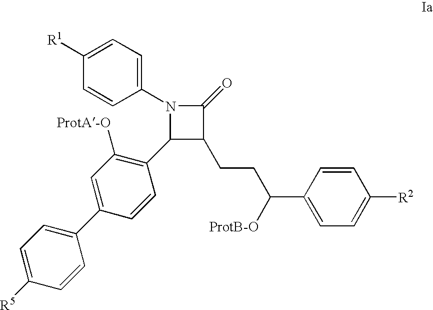

are prepared by reacting a compound of formula IIb

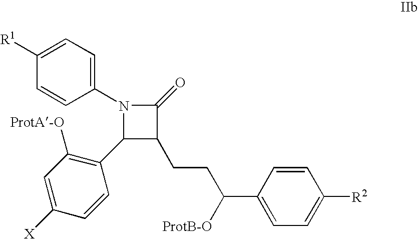

with a compound of formula III

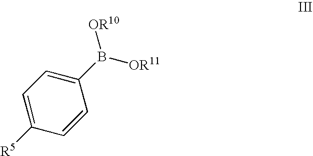

wherein R¹⁰ and R¹¹ are independently selected from H and (C₁-C₆) alkyl, or R¹⁰ and R¹¹ together form a 5-6 membered ring. Alternatively, one may react a compound of formula IIa

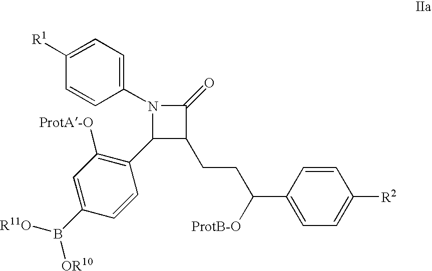

with a compound of formula XX

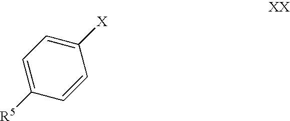

In these processes and compounds, R¹ and R² are chosen from H, halogen, —OH, and methoxy. R¹⁰ and R¹¹ together may form a 5-6 membered ring, for example:

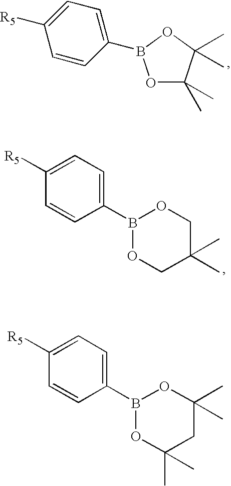

In certain embodiments, R¹ is hydrogen and R² is fluorine and R¹⁰ and R¹¹ together form a dioxaborole. The process for ADG is an example of such an embodiment.

ProtA-O— is a protecting group for a phenol chosen from protecting groups in Greene and Wuts, Chapter 3, that do not require removal with strong acid or base. Examples of such groups include oxymethyl ethers [e.g. MOM and 2-(trimethylsilyl)ethoxymethyl (SEM)], allyl ethers [e.g. allyl ether and 2-methylallyl ether], tertiary alkyl ethers [e.g. t-butyl ether], benzyl ethers [e.g. benzyl ether and various benzyl ether derivatives having substitution on the phenyl ring] and silyl ethers [e.g. trimethylsilyl, t-butyldimethylsilyl, and t-butyldiphenylsilyl].

ProtB-O— is HO— or a protecting group for a benzylic alcohol. For many reactions, including some illustrated below, it is unnecessary to protect the hydroxyl and in these cases, ProtB-O— is HO—. When a protecting group is desired, it is chosen from protecting groups in Greene and Wuts, Chapter 1, pages 17-86, the removal of which does not require strong acid. Examples include an oxymethyl ether, a tetrahydropyranyl or tetrahydrofuranyl ether, methoxycyclohexyl ether, a methoxybenzyl ether, a silyl ether and an ester [e.g. acetyl or benzoyl].

R⁵ is a sugar or a protected sugar. As discussed above, sugar encompasses any carbohydrate comprised of one or two saccharose groups as well as reduced sugars (alditols) such as glycitol. The protecting groups may be chosen from any of those well known in the carbohydrate art. Examples include benzyl ethers, silyl ethers [e.g. trimethylsilyl] and acyl esters [e.g. acetyl].

X is chosen from iodine, bromine, chlorine, toluenesulfonyl, methanesulfonyl and trifluoromethanesulfonyl.

In certain embodiments, ProtA-O— is chosen from methoxymethyl ether, t-butyl ether and benzyl ether; ProtB-O— is chosen from HO—, t-butyldimethylsilyl ether and tetrahydropyranyl ether; and III is

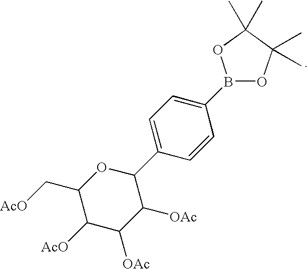

The reaction is brought about in the presence of a phosphine, a palladium salt and a base, for example triphenylphosphine or tri(o-tolyl)phosphine, PdCl₂ and an aqueous solution of an alkali metal hydroxide or carbonate. In one embodiment, R¹ is hydrogen; R² is fluorine; X is bromine; ProtA-O— is benzyl ether; and ProtB-O— is HO—.

Palladium catalysts include palladium acetate, palladium chloride, palladium bromide, palladium acetylacetonate, bis(tri-o-tolyl)phosphine palladium dichloride, bis(triphenylphosphine)palladium dichloride, tetrakis(triphenylphosphine)palladium [(Ph₃P)₄Pd], tris(dibenzylidene-acetone)palladium [(dba)₃Pd₂]and bis(dibenzylideneacetone) palladium [(dba)₂Pd]. In the formation of XII from XV, a phosphine ligand has been found advantageous. Ligands for the reaction with the diboron species may be 1,1′-bis(di-o-tolylphosphino)ferrocene (DTPF); 1,1′-bis(diphenylphosphino)ferrocene (DPPF); 1 -di-t-butylphosphino-2-methylaminoethyl ferrocene; [2′-(diphenylphosphino)[1,1′-binaphthalen]-2-yl]diphenylphosphine oxide (BINAP) and 2,2′-bis(di-p-tolylphosphino)-1,1′-binaphthyl (tol-BINAP) and trialkyl or triarylphosphines, such as tri-t-butylphosphine, tricyclohexyl phosphine, triphenylphosphine and (tri-o-tolyl)phosphine.

After the compound of formula I is synthesized, the protecting groups are cleaved under appropriate conditions to produce the corresponding compounds having a free phenol, free alcohol and/or free sugar/polyol. When the protecting group is, for example, benzyl, hydrogenolysis may be employed for deprotection; when the protecting group is, for example, t-butyldimethylsilyl, tetrabutylammonium fluoride may be employed for deprotection; when the protecting group is, for example, acetate, hydrolysis with aqueous base or methanolysis in the presence of fluoride anion may be employed for deprotection.

Thus, for example, one may prepare

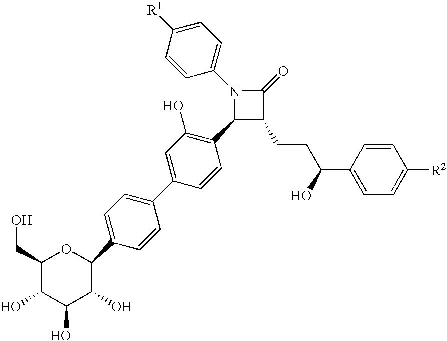

by reacting an azetidinone of formula

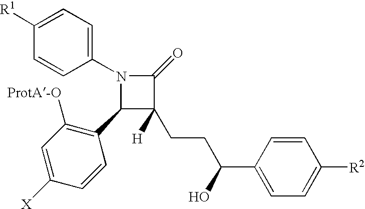

with a dioxaborole of formula

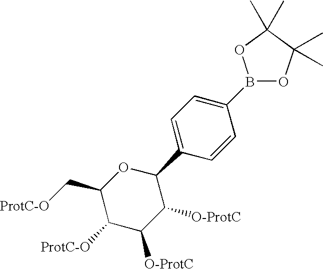

and deprotecting. In this example, ProtC-O— is a protecting group for a sugar alcohol chosen from a benzyl ether, a silyl ether and an ester. In a particular embodiment, one may react an azetidinone of formula

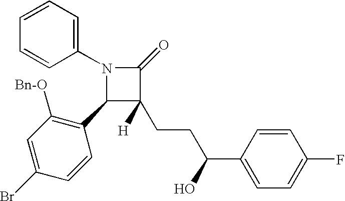

with a dioxaborole of formula

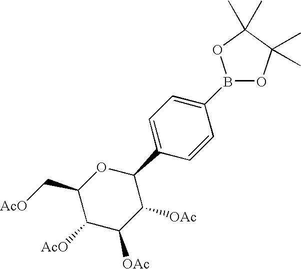

and deprotect. Deprotection of Prot′A (benzyl) is accomplished by catalytic hydrogenolysis and deprotection of ProtC (acetyl) is accomplished by hydrolysis with aqueous base or methanolysis in the presence of fluoride anion.

The compound of structure II may be synthesized by

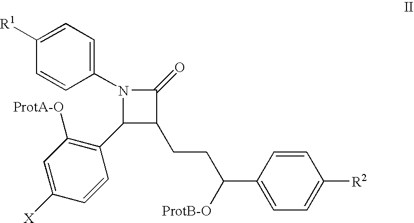

cyclizing a compound of formula IV

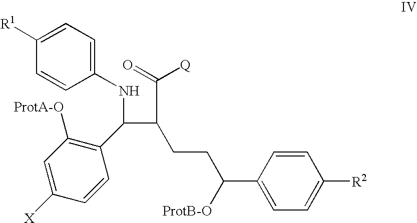

wherein Q is a chiral auxiliary attached at nitrogen. The chiral auxiliary may be chosen from single enantiomers of cyclic and branched nitrogen-containing moieties possessing at least one chiral center. In a specific embodiment, IIi:

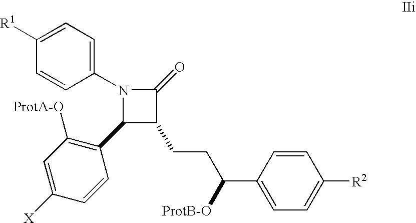

may be made by cyclizing a compound of formula IVi

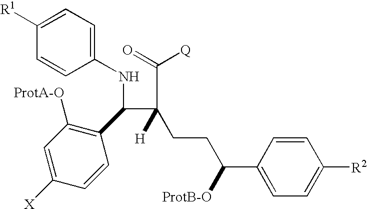

Examples of such chiral auxiliaries include triphenyl glycol:

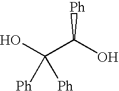

[see Braun and Galle, Synthesis 1996, 819-820], as well as the class of chiral nitrogen heterocycles:

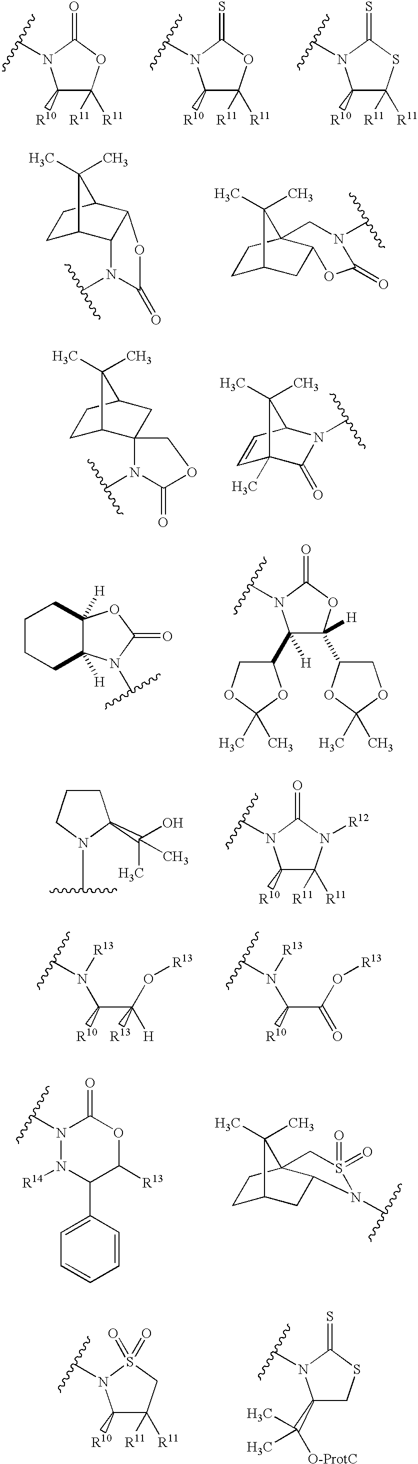

In these compounds, R¹⁰ is phenyl, benzyl, isopropyl, isobutyl or t-butyl; R¹¹ is hydrogen, methyl or ethyl; or R¹⁰ and R¹¹ together can form a cycle; R¹² is hydrogen, methyl or ethyl; R¹³ is hydrogen or methyl; R¹⁴ is methyl, benzyl, isopropyl, isobutyl or t-butyl; ProtC is methoxyoxymethyl (MOM), 2-(trimethylsilyl)ethoxymethyl (SEM), allyl or silyl [e.g. trimethylsilyl, t-butyldimethylsilyl, phenyldimethylsilyl]; and the wavy line indicates the bond by which the auxiliary is attached to the carbonyl of the parent. In one embodiment, the chiral auxiliary is

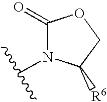

and R⁶ is phenyl or benzyl.

In one embodiment the precursor of the β-lactam is

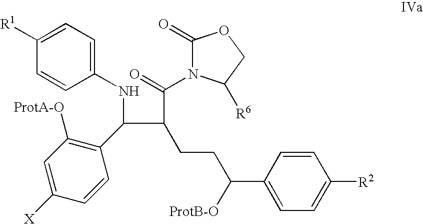

wherein R⁶ is phenyl or benzyl.

In one embodiment, in which ProtA-O— is methoxymethyl ether, allyl ether, t-butyl ether, silyl ether or benzyl ether and ProtB-O— is a silyl ether or tetrahydropyranyl ether, the cyclization is accomplished with N,O-bistrimethylsilylacetamide and a source of fluoride ion, such as tetrabutylammonium fluoride. The cyclization may also be carried out using a strong base, such as a metal hydride (e.g. sodium hydride, potassium hydride, lithium hydride).

The compound of formula IVi

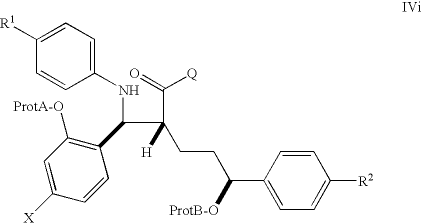

may be obtained by reacting a compound of formula V

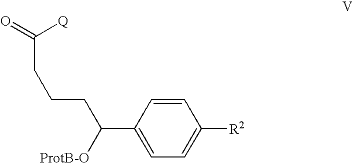

with a compound of formula VI

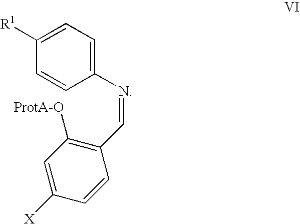

In one embodiment, compound of structure IVai

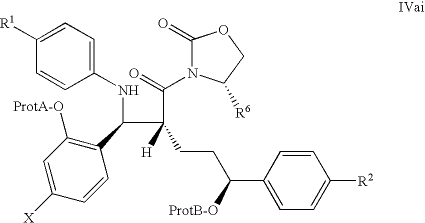

is produced by the sequential steps of
a. reacting a compound of formula Vai

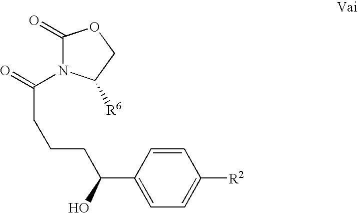

with a trialkylhalosilane in the presence of a base, such as an organic tertiary amine, followed by
b. a Lewis acid, particularly a halide of a Group 3, 4, 13 or 14 metal, such as titanium tetrachloride;
followed by
c. a compound of formula VI

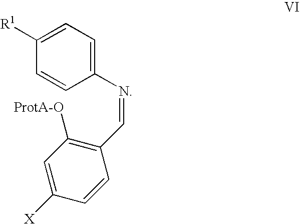

If the β-aminoacyloxazolinone component is protected (i.e. a compound of formula V in which ProtB-O is other than OH), “step a” can be omitted.

In another embodiment, a compound of formula

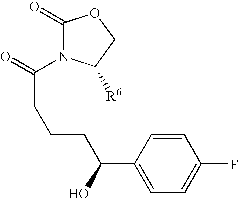

is reacted with trimethylclhlorosilane in the presence of a tertiary amine to provide a silyl-protected benzyl alcohol, and the silyl-protected benzyl alcohol is reacted with titanium tetrachloride and an imine of formula

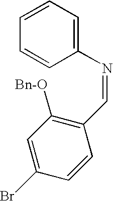

to provide a compound of formula

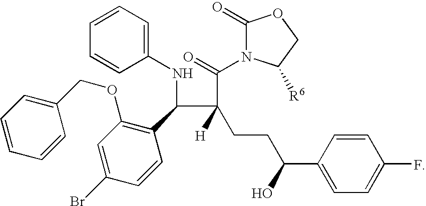

After the reaction of the silyl-protected benzyl alcohol with titanium tetrachloride and an imine, the product is isolated as a mixture in which the benzyl alcohol remains partly protected as the trimethylsilyl ether and partly deprotected to hydroxyl. The mixture can be converted entirely to the benzyl alcohol shown in the structure above by acid hydrolysis of the trimethylsilyl group and used in the next step or alternatively the mixture can be taken forward to the cyclization because the first part of the next step involves silylating the benzyl alcohol with N,O-bistrimethylsilylamide. Acid hydrolysis is preferred when the P-aminoacyloxazolinone will be purified by chromatography.

The compounds of formula V may be prepared by the process described in U.S. Pat. No. 6,627,757, in which Q is

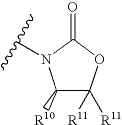

wherein R¹⁰ is phenyl and R¹¹ is hydrogen. Other chiral auxiliaries may be employed in the same fashion by replacing the N—H component

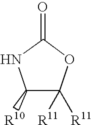

with any of the other appropriate Q groups described above.

The compounds of formula VI may be obtained by reacting a meta-substituted phenol with a source of formaldehyde forming a benzylic alcohol which undergoes Cannizzaro reaction to produce a benzaldehyde derivative, followed by Schiff base formation with an aniline of formula

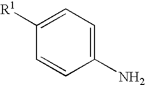

to produce a phenolic imine precursor to VI. The phenol is then protected under standard conditions appropriate for the chosen ProtA. For example, in the case in which ProtA is benzyl, the conditions are benzyl bromide and base. Sources of formaldehyde include paraformaldehyde, formaldehyde, trioxane and the like, all well known in the art. In the first step, the phenol reacts with formaldehyde in the presence of a magnesium salt, such as magnesium chloride, magnesium bromide or magnesium iodide, and a base. In the second step, the formylated phenol reacts with the aniline to provide the Schiff base VI.

Other routes to salicaldehydes may also be employed. Reaction of an appropriately substituted phenol in basic medium with formaldehyde (or chemical equivalent) will yield the corresponding salicylaldehyde. The intermediate, ortho-hydroxymethylphenol will be oxidized to the salicylaldehyde in situ. The reaction commonly employs ethyl magnesium bromide or magnesium methoxide (one equivalent) as the base, toluene as the solvent, paraformaldehyde (two or more equivalents) as the source of formaldehyde, and employs hexamethylphoramide (HMPA) or N,N,N′,N′-tetramethylethylenediamine (TMEDA). [See Casiraghi, G., et al., J. C. S. Perkin I, 1978, 318-321.] Alternatively the appropriately substituted phenol may react with formaldehyde under aqueous basic conditions to form the substituted ortho-hydroxybenzyl alcohol [See: a) J. Leroy and C. Wakselinan, J. Fluorine Chem., 40, 23-32 (1988); b) A. A. Moshfegh, et al., Helv. Chim. Acta., 65, 1229-1232 (1982)], and the resulting ortho-hydroxybenzyl alcohol can be converted to the salicylaldehyde by an oxidizing agent such as manganese (IV) dioxide in a solvent such as methylene chloride or chloroform [See R-G. Xie, et al., Synthetic Commun. 24, 53-58 (1994)].

An appropriately substituted phenol can be treated under acidic conditions with hexamethylenetetramine (HMTA) to prepare the salicyladehyde. This is well known as the Duff Reaction. [See Y. Suzuki, and H. Takahashi, Chem. Pharm. Bull., 31, 1751-1753 (1983)]. The Duff reaction commonly employs acids such as acetic acid, boric acid, methanesulfonic acid, or trifluoromethanesulfonic acid. The source of formaldehyde commonly used is hexamethylenetetramine.

One may also employ the Reimer-Tiemann reaction, in which an appropriately substituted phenol will react under basic conditions with chloroform to yield a substituted salicylaldehyde. [See Cragoe, E. J, Schultz, E. M, U.S. Pat. No. 3,794,734 (1974)].

The formylation of the dilithium salt of a phenol with a formamide [see Talley and Evans, J. Org. Chem. 49, 5267-5269 (1984)] also provides salicaldehydes. The disclosures of all the foregoing salicaldehyde syntheses are incorporated herein by reference.

The compounds of formula III

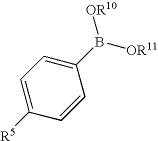

may be prepared according to the method shown in Scheme 6 for a specific embodiment XII, in which R¹⁰ and R¹¹ form a dioxaborole and X′ is chlorine. The scheme and supporting experimental description are noteworthy in that borate esters are not commonly made from aryl chlorides. In the present instance, a high yield is obtained. It appears to result from a combination of phosphine ligand and palladium catalyst and the use of high temperatures (>100° C.). The reaction of silylated lactone CC1 with Grignard goes in good yield, whereas the corresponding lithium reagent provides barely quantifiable product.

Also within the scope of the invention are two groups of compounds useful as intermediates in the processes described herein. The first of these are the (4-substituted phenyl)glycitols. The class of phenylglycitols may be further broken down to phenylglycitols of formula VIIa and those of IIIa.

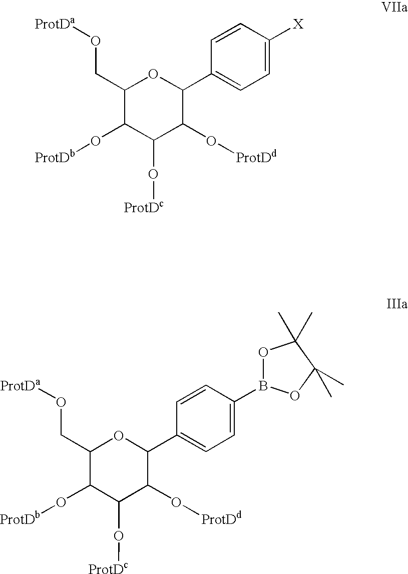

Phenylglycitols of formula VIIa are precursors to those of IIIa. The phenylglycitols IIIa are, of course, a subset of III in which R⁵ is a protected glycitol. A subgenus of VIIa is

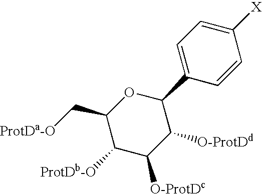

wherein
X is chosen from iodine, bromine, chlorine, toluenesulfonyl, methanesulfonyl and trifluoromethanesulfonyl; and
ProtD^{a}, ProtD^{b}, ProtD^{c} and ProtD^{d} are hydrogen or protecting groups for a sugar chosen independently from benzyl, silyl, acyl, ketal, acetal, methoxymethyl, 2-(trimethylsilyl)ethoxymethyl, allyl, 2-methylallyl and t-butyl. In one embodiment, when X is chlorine, ProtD^{a}, ProtD^{b}, ProtD^{c} and ProtD^{d} are not acetyl. In another embodiment, X is chlorine and ProtD¹, ProtD^{b}, ProtD^{c} and ProtD^{d} are acetyl.

The second novel class of compounds useful as intermediates in the processes described herein is the imines of formula VI

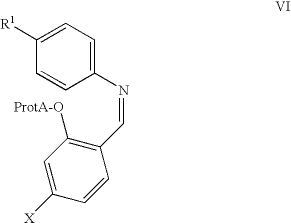

When ProtA- is benzyl, X is bromine and R¹ is H, the compound is solid and greater than 95% pure.

Exemplary processes that fall within the scope of the invention are illustrated in the schemes below. These schemes also illustrate the interrelatedness of the processes and intermediates. In the schemes that follow, solid arrows indicate reactions that are described in the examples; dashed arrows indicate reactions that are not exemplified.

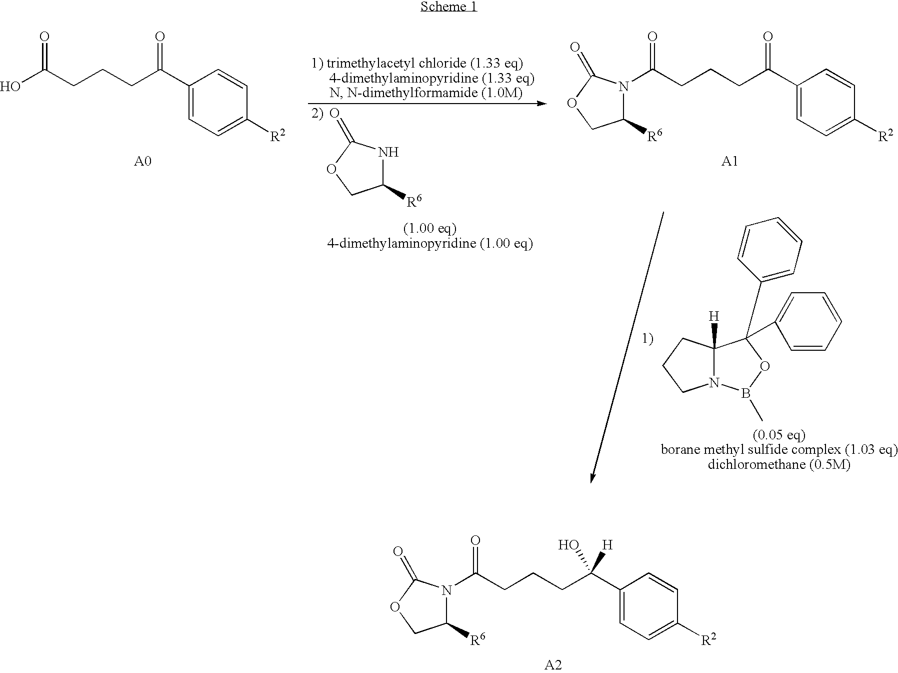

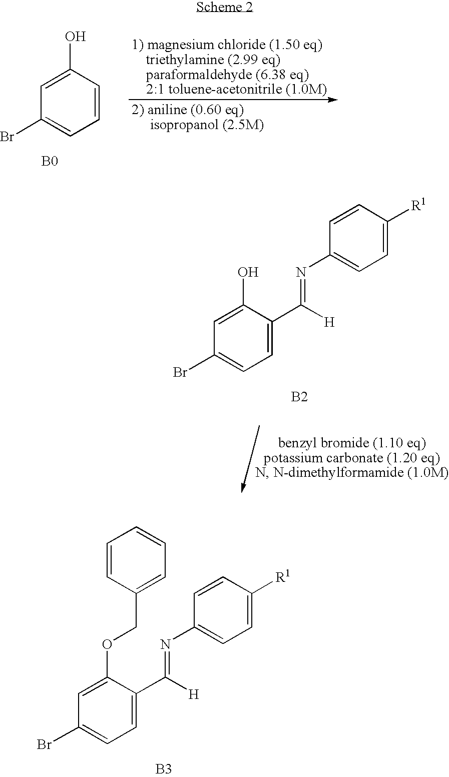

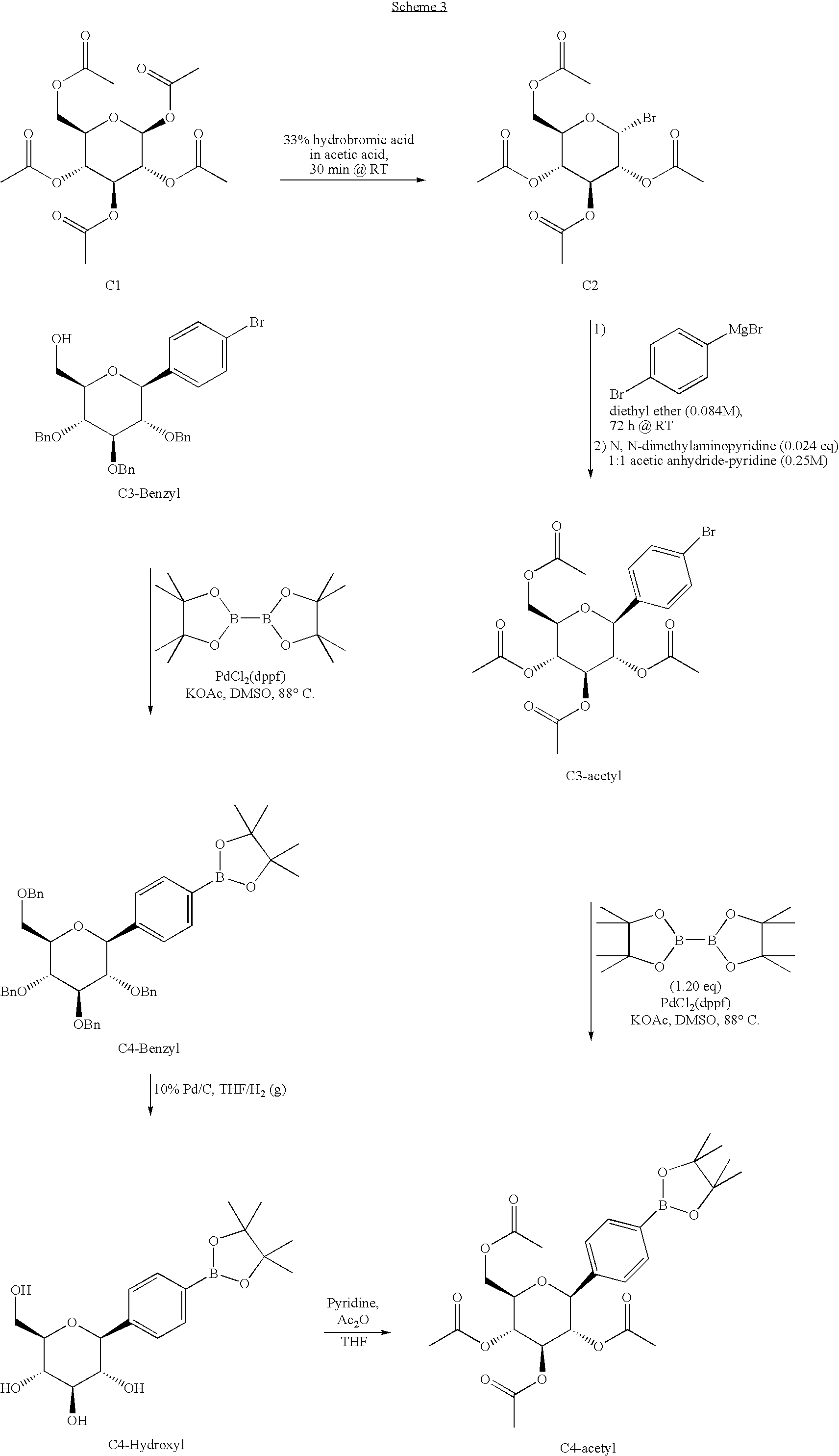

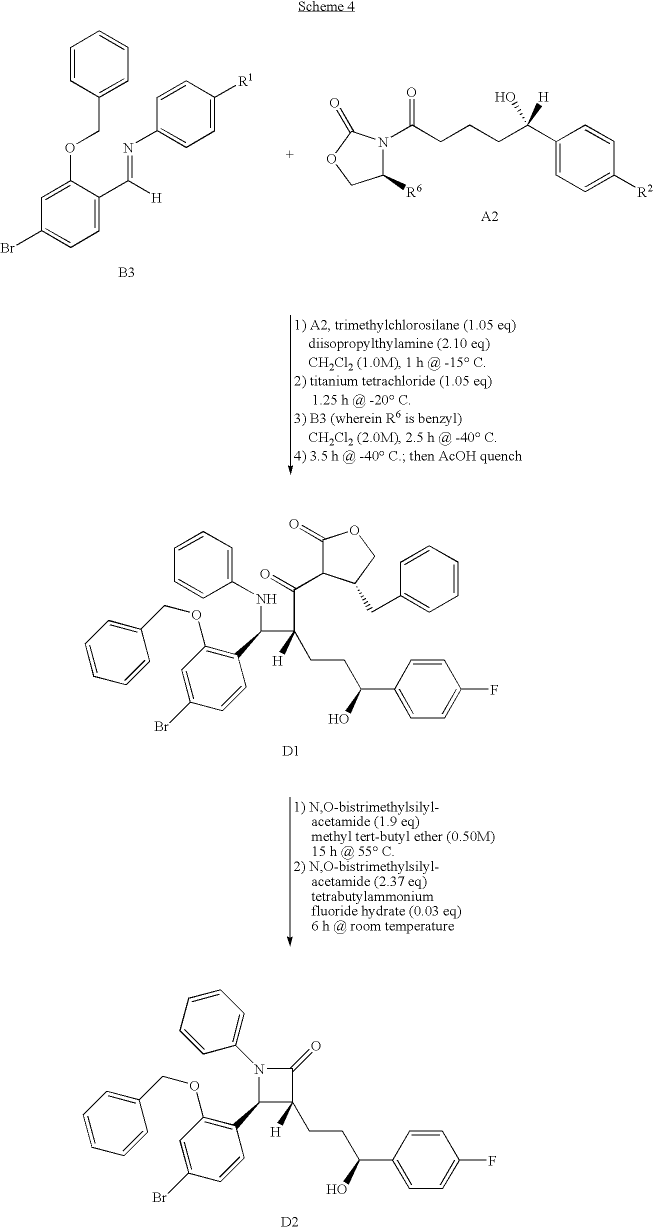

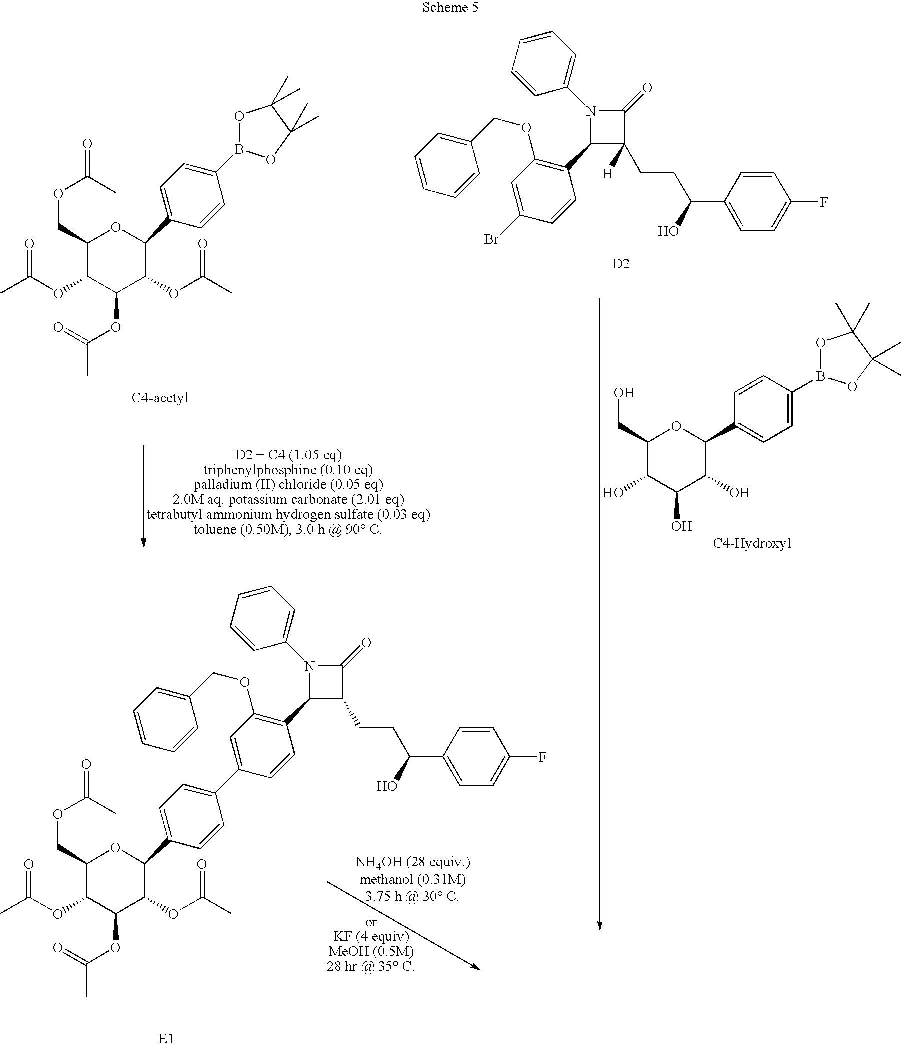

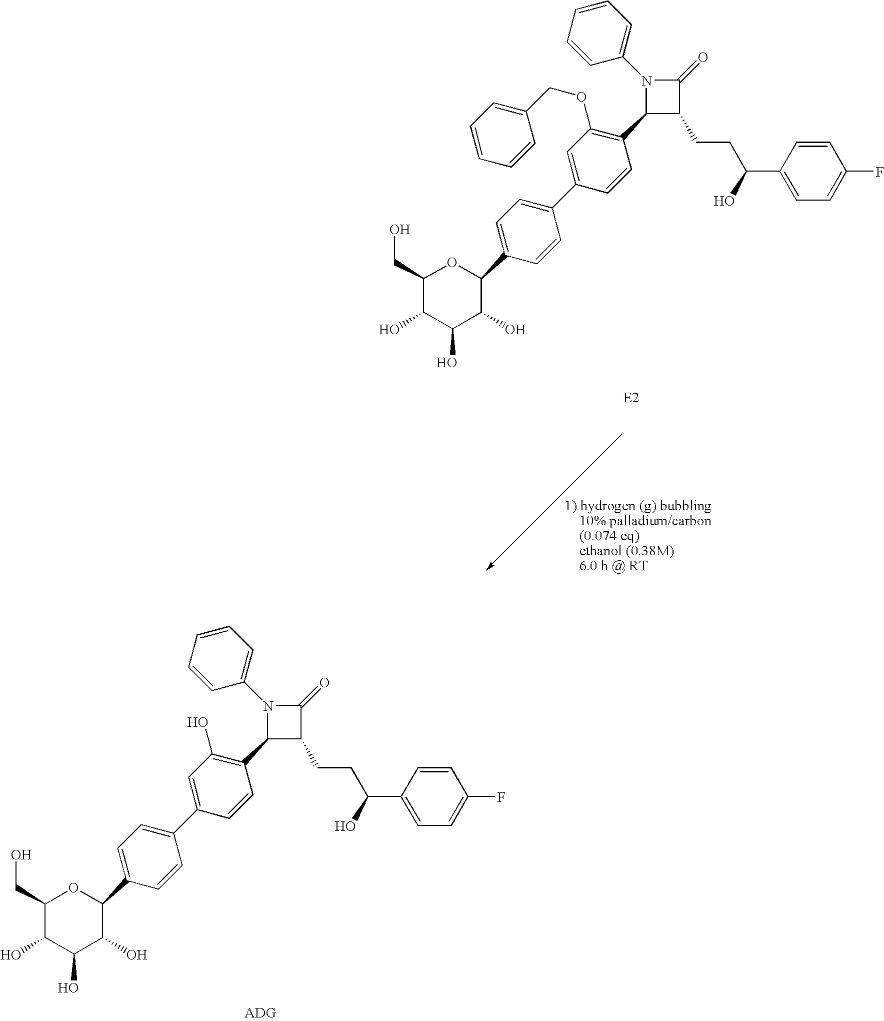

In the foregoing Scheme 5, the reaction of D2 with C4 to produce E1 is not as clean when carried out using triphenylphosphine as it is when tri(ortho-tolyl)phosphine and palladium chloride are employed. The triphenylphosphine-palladium chloride catalyzed reaction produces, in addition to E1, two identifiable impurities, E11 and E12:

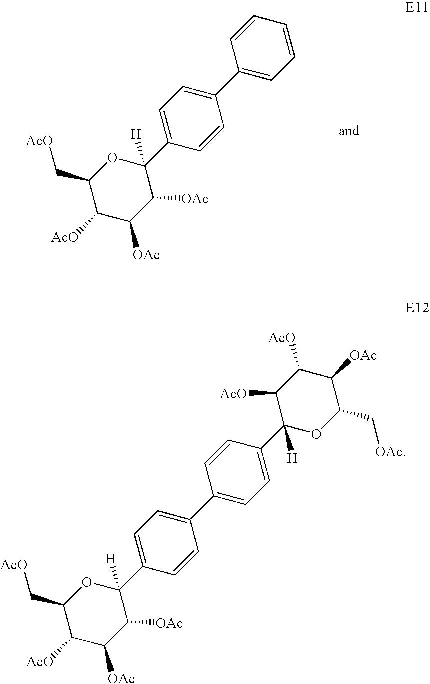

The use of tri(ortho-tolyl)phosphine CAS#[6163-58-2] and palladium (II) chloride in the presence of aqueous potassium carbonate and tetrabutylammonium hydrogensulfate in the synthesis described below as Step 10, produces (1S)-2,3,4,6-tetra-O-acetyl-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E1) in greater than 99% chemical purity containing less than 1% in combination of E11 and E12.

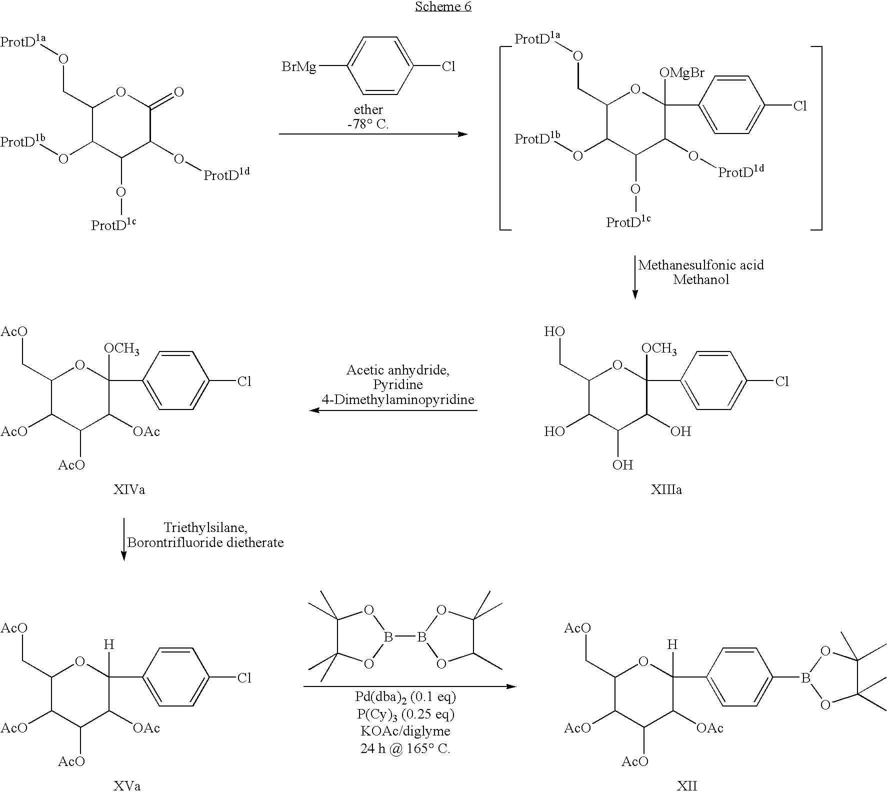

### Step 1. Preparation of (4S)-4-benzyl-3-[5-(4-fluorophenyl)-5-oxopentanoyl]-1,3-oxazolidin-2-one (A1)

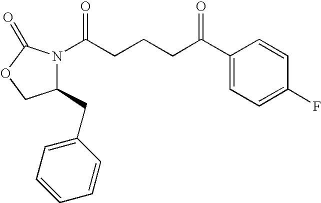

5-(4-Fluorophenyl)-5-oxopentanoic acid (372.0 g, 1.77 mol) and 4-dimethylamino-pyridine (286.9 g, 2.35 mol) were dissolved in N,N-dimethylformamide (1770 mL, 1.0 M) to afford a copious white precipitate suspended in solution. The reaction was cooled to 6° C. (ice/water bath), trimethylacetyl chloride (290 mL, 2.35 mol) was added quickly drop-wise over 17 min to afford a pale yellow mixture. The rate of addition was controlled in order to keep the temperature below 8.5° C. The mixture was stirred for 1 h at 9° C. (ice/water bath) then for 2 h at 20° C. (colorless solution with copious white thick precipitate). The mixture was charged with (S)-benzyl-2-oxazolidinone (313.5 g, 1.77 mol) and 4-dimethylaminopyridine (216.4 g, 1.77 mol) both as solids to afford a bright yellow colored suspension. The reaction was stirred at 27° C. for 3.3 h. The pale olive colored solution was poured into water (4300 mL) while stirring vigorously (an exotherm was detected to 39° C.), transferred with water (1000 mL) and stirred at room temperature for 2 h to afford a pale orange-brown solution with an off-white precipitate. The compound was filtered, transferred with water (2×300 mL), washed with water (400 mL) and air dried for 1.5 h to afford an off-white moist clumpy powder. The material was crystallized from isopropanol (2600 mL, 4.0 mL/g theoretical yield) by heating to near reflux to afford a dark golden yellow colored solution. The mixture was cooled slowly from 81° C. to 74° C. in 20 min, a seed crystal was added and crystals began to precipitate. The mixture was cooled slowly to room temperature over 11 h, cooled to 2° C. in an ice/water bath and stirred for 3 h. The crystals were filtered, transferred with cold mother liquor (350 mL), washed with cold isopropanol (2×350 mL), air dried and vacuum dried to constant weight to afford (4S)-4-benzyl-3-[5-(4-fluorophenyl)-5-oxopentanoyl]-1,3-oxazolidin-2-one (A1) (510.6 g, 78 % yield) as a white crystalline solid; m.p. 113.4±1.2° C.; R_{f} 0.37 (1:2 ethyl acetate-hexane); HPLC purity 99.7 A % (96.4 A % by NMR); ¹H NMR (300 MHz, CDCl₃) δ 8.03-7.98 (m, 2H), 7.37-7.19 (m, 5H), 7.14 (t, J=8.7 Hz, 2H), 4.72-4.64 (m, 1H), 4.25-4.15 (m, 2H), 3.32 (dd, J=13.3, 3.4 Hz, 1H), 3.12-3.01 (m, 4H), 2.78 (dd, J=13.3, 9.6 Hz, 1H), 2.15 (quint., J=7.2 Hz, 2H), ppm.

In the synthesis of (4S)-4-benzyl-3-[5-(4-fluorophenyl)-5-oxopentanoyl]-1,3-oxazolidin-2-one (A1), two side products are formed:

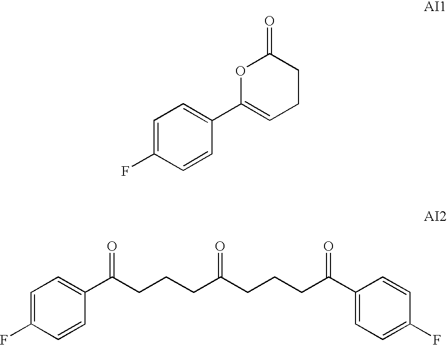

The first of these, AI1, can be reduced with hydrogen in the presence of a chiral catalyst to produce AI4

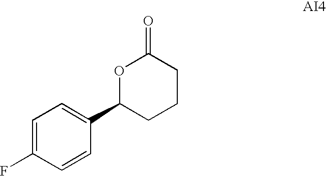

which can be utilized in the synthesis of D2 using the procedure described in PCT WO2004 099132. Although AI1 and AI2 were isolated by chromatography from the reaction described above, if one wishes to make AI1 directly, one can react 5-(4-fluorophenyl)-5-oxopentanoic acid with oxalyl chloride. The second by-product, AI2, if not removed, is subsequently reduced to AI3

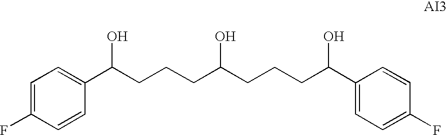

in the following step. It then co-crystallizes with A2 from toluene/alkane solvents and remains an impurity in A2. It can be removed from A2 by crystallization from isopropanol/alkane. The analytical assessment of the products is by TLC or HPLC with the following results:
A0—R_{f} 0.08 (1:2 ethyl acetate-hexane); HPLC R_{T} 3.7 min;
A1—R_{f} 0.37 (1:2 ethyl acetate-hexane); HPLC R_{T} 7.4 min;
A2—R_{f} 0.14 (1:2 ethyl acetate-hexane); HPLC R_{T} 6.5 min;
AI1—R_{f} 0.50 (1:2 ethyl acetate-hexane); HPLC R_{T} 5.5 min;
AI2—R_{f} 0.38 (1:2 ethyl acetate-hexane); HPLC R_{T} 7.6 min;
AI3—R_{f} 0.43 (2:1 ethyl acetate-hexane); HPLC R_{T} 5.4 min.
HPLC on Waters Xterra® MS C₁₈ (3.0×150 mm), 5 μm at 35° C.
Mobile Phase (A): 0.1% Formic Acid in Water (HPLC grade)
Mobile Phase (B): Acetonitrile (HPLC grade)
Gradient Program: 25% B—initial conditions
   25% to 100% B—11 min
   100% to 25% B—0.4 min
   25% B—3.6 min (flow increase to 1.75 mL/min)
Detection: 254 nm
Flow Rate: 1.0 mL/min
Run Time: 15 min
AI1 6-(4-fluorophenyl)-3,4-dihydro-2H-pyran-2-one. ¹H NMR (CDCl₃/300 MHz) 7.54(dd, 2H, J=5.1, 9.0 Hz), 7.01(dd, 2H, J=9.0, 9.0 Hz), 5.72(t, 1H, J=4.8 Hz), 2.68-2.63(m, 2H), 2.51-2.47(m, 2H). Mass spectrum, M+H=193.
AI2 1,9-bis(4-fluorophenyl)nonane-1,5,9-trione, mp 97.1±0.7° C. ¹H NMR (CDCl₃/300 MHz) 7.92(dd, 4H, J=5.4, 9.0 Hz), 7.06(dd, 4H, J=9.0, 9.0 Hz), 2.92(t, 4H, J=6.9 Hz), 2.49(t, 4H, J=6.9 Hz), 1.95(sept, 4H, J=6.9 Hz). Mass spectrum, M+H=359.
AI3 (1S,9S)-1,9-bis(4-fluorophenyl)nonane-1,5,9-triol. ¹H NMR (CDCl₃/300 MHz) 7.24(dd, 4H, J=5.4, 8.4 Hz), 6.98(dd, 4H, J=8.4, 8.4 Hz), 4.60(m, 2H), 3.52(m, 1H), 3.20-2.60(m, 2H), 1.80-1.20(m, 10H). Mass spectrum, M+H=365.

### Step 2. Preparation of (4S)-4-benzyl-3-[(5s-5-(4-fluorophenyl)-5-hydroxypentanoyl]-1,3-oxazolidin-2-one (A2)

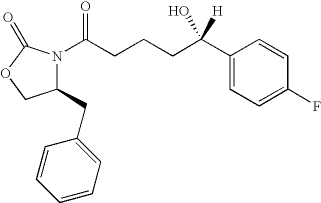

(4S)-4-Benzyl-3-[5-(4-fluorophenyl)-5-oxopentanoyl]-1,3-oxazolidin-2-one (A1) (500.0 g, 1.35 mol) was dissolved in dichloromethane (2700 mL, 0.5 M). The mixture was cooled to 4° C. (ice/brine bath), stirred for 40 min and charged with 1.0 M (R)-1-methyl-3,3-diphenyltetrahydro-3H-pyrrolo[1,2-c] [1,3,2] oxazaborole in toluene (68 mL, 0.068 mol). After 10 min, borane-methyl sulfide complex (132 mL, 1.39 mol) was added drop-wise via addition funnel over 25 min (an exotherm was detected to −2.7° C.). The reaction was maintained between 0 and −6° C. with stirring for 3.0 h. The reaction was quenched by slow addition of methanol (275 mL, 6.79 mol) over 15 min (an exotherm was detected to 10° C.), 6% aqueous hydrogen peroxide (1150 mL, 2.02 mol) over 5 min and 1.0 M aqueous sulfuric acid (810 mL, 0.81 mol) over 15 min (an exotherm was detected to 17° C.) respectively via addition funnel. The reaction was stirred at room temperature for 60 min, poured into a separatory funnel, the organic layer was separated and the aqueous layer was extracted with dichloromethane (2000 mL). The first organic layer was washed with water (1500 mL) and brine (1500 mL). These aqueous layers were backed extracted with the second organic layer. The combined organic layers were partially concentrated, dried over sodium sulfate, filtered through Celite®, concentrated and crystallized from isopropanol-heptane (2000 mL, 1:1 isopropanol-heptane; 4.0 mL/g theoretical yield). The clear viscous residue was warmed to 42° C. (to make a homogeneous solution), cooled slowly to 35° C., held at this temperature for 12 h, cooled slowly to room temperature over 3 h, cooled to 0 to −5° C. (ice/brine bath) and stirred for 2 h. The crystals were filtered, transferred with cold mother liquor (250 mL), washed with cold 1:2 isopropanol-heptane (2×400 mL), air dried and vacuum dried to constant weight to afford (4S)-4-benzyl-3-[(5S)-5-(4-fluorophenyl)-5-hydroxypentanoyl]-1,3-oxazolidin-2-one (A2) (445.8 g, 89% yield) as a white crystalline solid; m.p. 75.4±0.6° C.; R_{f} 0.12 (1:2 ethyl acetate-hexane); HPLC purity 98.9A %; ¹H NMR (300 MHz, CDCl₃) δ 7.37-7.24 (m, 5H), 7.19 (d, J=7.3 Hz, 2H), 7.02 (t, J=8.9 Hz, 2H), 4.72-4.61 (m, 2H), 4.21-4.13 (m, 2H), 3.27 (dd, J=13.2, 3.0 Hz, 1H), 2.99-2.94 (m, 2H), 2.74 (dd, J=13.2, 9.6 Hz, 1H), 2.27 (br s, 1H), 1.88-1.66 (m, 4H) ppm; [α]_{C}²³+72.9° (c 7.0, methanol).

### Step 3. Preparation of 5-bromo-2-[(E)-(phenylimino)methyl]phenol (B2)

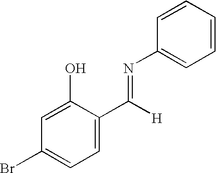

3-Bromophenol (498.5 g, 2.88 mol) was dissolved in a mixture of 2:1 toluene-acetonitrile (3000 mL, 0.96 M). To this solution was added triethylamine (1200 mL, 8.61 mol) via funnel. Magnesium chloride (412.7 g, 4.33 mol) was added in one portion as a solid (an exotherm was detected to 55° C.) to afford a bright yellow solution with copious white precipitate. Paraformaldehyde (345 g, 11.5 mol) was added as a suspension in acetonitrile (300 mL) while the temperature of the solution was 45° C. (an exotherm was detected to 78.6° C.). The temperature of the yellow-orange slurry was maintained at 80±3° C. for 1.5 h while the by-product (methanol) was distilled off (white precipitate was observed depositing in the distillation apparatus and reflux condensers). A second portion of paraformaldehyde (100 g, 3.33 mol) was added as a suspension in acetonitrile (200 mL). The mixture was heated for 2 h and another portion of paraformaldehyde (107 g, 3.56 mol) was added as a suspension in acetonitrile (200 mL). The mixture was stirred for 2.5 h at 80±4° C. After a total of 6 h and 6.4 equivalents total of paraformaldehyde had been added, the mixture was quenched with cold 2.5 N aqueous hydrochloric acid (6000 mL, 15 mol) added over 5 min. The mixture was stirred to room temperature for 60 min to afford a biphasic solution with a dull yellow top layer and dark orange bottom layer. The solution was diluted with 4:1 heptane-ethyl acetate (1000 mL), agitated and the layers separated. The aqueous layer was extracted with 4:1 heptane-ethyl acetate (2×1500 mL). Each organic layer was washed with the same portion of water (1800 mL) and brine (1800 mL). All the organic layers were combined, partially concentrated, dried over sodium sulfate, filtered through Celite® and concentrated to afford 2-hydroxy-4-bromobenzaldehyde as a dark golden-orange viscous oil; R_{f} 0.54 (1:4 ethyl acetate-hexane); HPLC purity 60 A %.

Crude 2-hydroxy-4-bromobenzaldehyde was dissolved in isopropanol (1000 mL, 1.26 mL/g theoretical yield, 2.5 M) and the mixture was heated to 75° C. Aniline (157 mL, 1.72 mol) was added to afford a bright orange solution and the mixture was left to cool slowly to room temperature (an exotherm was detected to 83° C. as imine crystallized from solution.) The mixture was stirred at room temperature for 12 h. The crystals were filtered, transferred with isopropanol (500 mL), washed with isopropanol (500 mL), air dried under a heavy stream of dry nitrogen gas and vacuum dried to constant weight to afford 5-bromo-2-[(E)-(phenylimino)methyl]phenol (B2) (347.4 g, 44% yield over two steps) as a bright yellow crystalline solid; m.p. 129.1±0.1° C.; R_{f} 0.65 (1:4 ethyl acetate-hexane); NMR purity >99 A %; ¹H NMR (300 MHz, CDCl₃) δ 8.59 (s, 1H), 7.47-7.40 (m, 2H), 7.33-7.22 (m, 5H), 7.08(dd, J=8.2, 1.8 Hz, 1H), 1.57 (br s, 1H) ppm.

### Step 4. Preparation of N-{(1E)-[2-(benzyloxy)-4-bromophenyl]methylene}-N-phenylamine (B3)

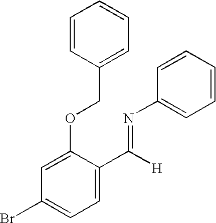

5-Bromo-2-[(E)-(phenylimino)methyl]phenol (B2) (310.9 g, 1.13 mol) was dissolved in anhydrous N,N-dimethylformamide (1100 mL, 1.0 M). Solid potassium carbonate (186.7 g, 1.35 mol) was added followed benzyl bromide (147.1 mL, 211.5 g, 1.24 mol) via syringe. The reaction was stirred under nitrogen for 4 h at room temperature and quenched with water (2000 mL) (an exotherm was detected to 40° C.). A yellow precipitate formed and the mixture was stirred for 1 h at room temperature. The solution was filtered and transferred with water (500 mL) and air dried under a heavy stream of dry nitrogen gas for 15 min. Crude solid was dissolved in isopropanol (1250 mL, 3.0 mL/g theoretical yield, 0.9 M) and the mixture was heated to 83° C. to afford a clear dark yellow solution which was cooled slowly to room temperature. The mixture was stirred at room temperature for 12 h. The crystals were filtered, transferred with cold isopropanol (250 mL), washed with cold isopropanol (250 mL), air dried under a heavy stream of dry nitrogen gas and vacuum dried to constant weight to afford N-{(1E)-[2-(benzyloxy)-4-bromophenyl]methylene}-N-phenylamine (B3) (375.2g, 91% yield) as a light yellow crystalline solid; m.p. 100.2±0.2° C.; R_{f} 0.59 (1:4 ethyl acetate-hexane); NMR purity >99 A %; ¹H NMR (300 MHz, CDCl₃) δ 8.87 (s, 1H), 8.06 (d, J=8.2 Hz, 1H), 7.43-7.33 (m, 7H), 7.28-7.17 (m, 5H), 5.14 (s, 2H) ppm.

### Step 5. Preparation of (4S)-3-[(2R,58)-2-{(S)-anilino[2-(benzyloxy)-4-bromophenyl]methyl}-5-(4-fluorophenyl)-5-hydroxypentanoyl]-4-benzyl-1,3-oxazolidin-2-one (D1)

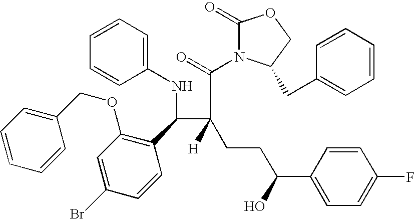

A 5-L three-necked flask was charged with (4S)-4-benzyl-3-[(5s)-5-(4-fluorophenyl)-5-hydroxypentanoyl]-1,3-oxazolidin-2-one (203.2 g, 0.547 mol) followed by addition of anhydrous dichloromethane (550 mL, 1.0 M) and N-ethyldiisopropylamine (200 mL, 148.4 g, 1.148 mol) via funnel. The reaction was cooled to −15° C. and trimethylchlorosilane (73.0 mL, 62.5 g, 0.575 mol) was added via cannula over 10 min (an exotherm was detected to −8° C.). The reaction was stirred for 1 h between −25° C. and −15° C. Titanium tetrachloride (63.0 mL, 109.0 g, 0.575 mol) was added drop-wise via addition funnel over 35 min to afford a deep reddish purple solution (an exotherm was detected to −10° C.). The mixture was stirred at −20±4° C. for 40 min, cooled to −40° C. and N-{(1E)-[2-(benzyloxy)-4-bromophenyl]methylene}-N-phenylamine (375.2 g, 1.024 mol) was added in dichloromethane (510 mL, 2.0 M) drop-wise slowly via addition funnel over 2.5 h. The reaction temperature was maintained between −45° C. and −31° C. The mixture was stirred for an additional 3.5 h, quenched by slow addition of glacial acetic acid (125 mL, 2.19 mol) over 15 min (the reaction temperature was maintained between −33° C. and −31° C.) and diluted with cold (10° C.) 15% aqueous dl-tartaric acid solution (2200 mL) (an exotherm was detected to 0° C.). This mixture was stirred to 17° C. over 2 h, diluted with dichloromethane (1000 mL), poured into a separatory funnel and the layers were separated. The organic layer was washed with 10% saturated brine solution (2000 mL) and brine (1000 mL). The aqueous layers were re-extracted sequentially with 1:1 ethyl acetate-heptane (2×1500 mL) and the combined organic layers were concentrated to afford a viscous reddish residue and copious yellow precipitate. The mixture was diluted with 1:4 dichloromethane-heptane (1000 mL), filtered and the solid was washed with 1:4 dichloromethane-heptane (3×500 mL). The filtrate was concentrated and the residue was diluted with dichloromethane (600 mL) and loaded onto silica gel (700 mL). The mixture was purified by pad filtration (300 mL silica gel, dichloromethane (300 mL) and 15% ethyl acetate-dichloromethane (4000 mL)) to afford (4S)-3-[(2R,5S)-2-{(S)-anilino[2-(benzyloxy)-4-bromophenyl]methyl}-5-(4-fluorophenyl)-5-hydroxypentanoyl]-4-benzyl-1,3-oxazolidin-2-one (D1) as a viscous, dark yellow, oil, which was used as-is in Step 4. ¹H NMR (300 MHz, CDCl₃) δ 7.50 (dd, J=8.2, 1.5 Hz, 2H), 7.39-7.30 (m, 3H), 7.26-6.98 (m, 12H), 6.94 (t, J=8.6 Hz, 2H), 6.62 (t, J=7.3 Hz, 1H), 6.52 (d, J=8.6 Hz, 2H), 5.13 (s, 2H), 5.06 (d, J=6.5 Hz, 1H), 4.73 (dd, J=13.8, 6.7 Hz, 1H), 4.64-4.57 (m, 1H), 4.49 (dd, J=7.3, 5.2 Hz, 1H), 4.12-4.04 (m, 2H), 3.01 (dd, J=13.4, 3.0 Hz, 1H), 2.39 (dd, J=13.4, 9.5 Hz, 1H), 1.84-1.51 (m, 6H) ppm.

### Step 6. Preparation of (3R,4S)-4-[2-(benzyloxy)-4-bromophenyl]-3-[(3S)-3-(4-fluorophenyl)-3-hydroxypropyl]-1-phenylazetidin-2-one (D2)

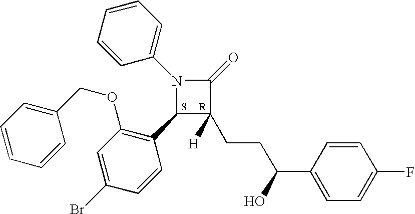

A 3-L three-necked flask was charged with semi-pure (4S)-3-[(2R,5S)-2-{(S)-anilino[2-(benzyloxy)-4-bromophenyl]methyl}-5-(4-fluorophenyl)-5-hydroxypentanoyl]-4-benzyl-1,3-oxazolidin-2-one (0.547 mol) in anhydrous tert-butyl methyl ether (1100 mL, 0.5 M) and N,O-bistrimethylsilylacetamide (250 mL, 1.012 mol, free of chlorotrimethylsilane) was added. The mixture was stirred at 55° C. for 15 h and then N,O-bistrimethylsilylacetamide (320 mL, 1.294 mol) was added followed by a catalytic amount of tetrabutylammonium fluoride trihydrate (4.62 g, 0.0177 mol) to afford a color change from bright yellow to pale golden yellow. The reaction was stirred at room temperature for 6 h and quenched with glacial acetic acid (1.0 mL, 0.018 mol). Hydrolysis of the silyl protecting groups is accomplished with 1.0 N aqueous hydrochloric acid (1100 mL) which was added drop-wise to avoid an exotherm (decompostion of the N,O-bistrimethylsilylacetamide with aqueous acid can be reactive). The bright yellow biphasic mixture was stirred for 1.5 h, poured into a separatory funnel, diluted with 1:1 ethyl acetate-heptane (1000 mL) and water (1000 mL), agitated, the layers were separated and the organic layer was washed with 5-25% aqueous sodium bisulfite, water (500 mL) and brine (500 mL). The two aqueous layers were back-extracted sequentially with one portion of 1:1 ethyl acetate-heptane (1000 mL) and the combined organic layers were concentrated. The residue was diluted with 1:1 heptane-dichloromethane (1000 mL), made into a slurry with silica gel (1000 mL) and purified by pad filtration (2000 mL silica gel, 10% (8000 mL), 20% (8000 mL), 30% (6000 mL) and 40% (4000 mL) ethyl acetate-hexane) to afford (3R,4S)-4-[2-(benzyloxy)-4-bromophenyl]-3-[(38)-3-(4-fluorophenyl)-3-hydroxypropyl]-1-phenylazetidin-2-one (D2) (251.2 g, 82%) as a pale dull yellow foam; HPLC purity 89 A %; NMR purity 85 A %. A portion of the residue (124.2 g) was purified by crystallization from warm 8% water-methanol (500 mL, 4.0 mL/g, theoretical yield). The crystals were filtered, washed with cold 10% water-methanol (200 mL), air dried and vacuum dried to constant weight to afford (3R,4S)-4-[2-(benzyloxy)-4-bromophenyl]-3-[(3S)-3-(4-fluorophenyl)-3-hydroxypropyl]-1-phenylazetidin-2-one (D2) (85.9 g, 77% recovery based the amount of desired compound in the crude starting material) as white crystalline needles; m.p. 113±0.5° C.; R_{f} 0.32 (1:2 ethyl acetate-hexane); HPLC purity >99 %; NMR purity >99%; ¹H NMR (300 MHz, CDCl₃) δ 7.41 (br s, 5H), 7.28-7.22 (m, 4H), 7.19-7.15 (m, 3H), 7.08-7.02 (m, 3H), 6.96 (t, J=8.7 Hz, 2H), 5.10 (dd, J=15.2, 11.2 Hz, 2H), 5.01 (d, J=2.4 Hz, 1H), 4.57-4.52 (m, 1H), 3.06-3.00 (m, 1H), 2.25 (d, J=3.8, 1H), 1.97-1.74 (m, 4H) ppm; [α]_{D}²³−12.3° (c 6.5, ethyl acetate).

Alternate Route to (3R,4S)-4-[2-(benzyloxy)-4-bromophenyl]-3-[(3S)-3-(4-fluorophenyl)-3-hydroxypropyl]-1-phenylazetidin-2-one (D2).

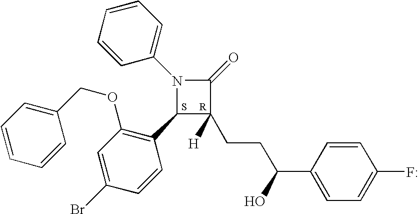

### Step 1A. Preparation of (4S)-4-phenyl-3-[5-(4-fluorophenyl)-5-oxopentanoyl]-1,3 -oxazolidin-2-one (A1 R⁶=phenyl)

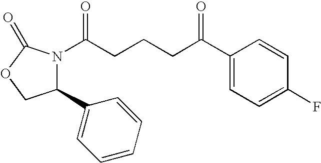

5-(4-Fluorophenyl)-5-oxopentanoic acid (21.02 g, 100.0 mmol) and 4 dimethylamino-pyridine (16.25 g, 133.0 mmol) were dissolved in N,N-dimethylformamide (100 mL, 1.0 M) to afford a copious white precipitate suspended in solution. The reaction was cooled to 2° C. (ice/water bath), and trimethylacetyl chloride (16.40 mL, 16.04 g, 133.0 mmol) was added drop-wise to afford a pale yellow mixture. The rate of addition was controlled in order to keep the temperature at or below 5° C. A heavy white precipitate was formed and the mixture was allowed to warm to room temperature and stirred for 1.5 h. The mixture was charged with (s)-(+)-4-phenyl-2-oxazolidinone (16.32 g, 100.0 mmol) and 4-dimethylaminopyridine (12.22 g, 100.0 mmol) both as solids to afford a yellow colored suspension. The reaction was stirred at 30° C. −35° C. for 2 h. An aliquot was removed for analysis by TLC and HPLC. The pale olive colored suspension was poured into water (400 mL) while stirring vigorously and cooling the mixture in an ice-brine bath, transferred with water (150 mL) and stirred with ice-cooling for 1.5 h to afford a solution with an off-white precipitate. The compound was filtered, transferred with water (2×25 mL), washed with water (50 mL) and air dried for 15 min to afford an off-white moist clumpy powder. The material was crystallized from isopropanol (58.0 mL; 1.6 mL/g theoretical yield) by heating to near reflux to afford a golden yellow colored solution. The solution was cooled slowly to room temperature over 12 h, a seed crystal was added and crystals began to precipitate. The mixture was cooled in an ice/water bath and stirred for 1 h. The crystals were filtered, transferred with cold isopropanol (2×10 mL), washed with cold isopropanol (25 mL), air dried and vacuum dried to constant weight to afford (4S)-4-phenyl-3-[5-(4-fluorophenyl)-5-oxopentanoyl]-1,3-oxazolidin-2-one (30.46 g, 85.7% yield) as a white crystalline solid; m.p. 91.0° C.; R_{f} 0.40 (1:2 ethyl acetate-hexane); HPLC R_{T} 7.02 min; HPLC purity 94%. ¹H NMR (300 MHz, CDCl₃) δ 7.93 (dd, J=5.4, 9.0 Hz, 2H), 7.28-7.42 (m, 5H), 7.10 (dd, J=8.5, 9.0 Hz, 2H), 5.43 (dd, J=3.7, 8.7 Hz, 1H), 4.70 (t, J=8.9 Hz, 1H), 4.28 (dd, J=3.7, 8.7 Hz, 1H), 3.05 (dt, J=1.2, 7.3 Hz, 2H), 2.97 (t, J=7.3, 2H), 2.05 (p, J=7.3 Hz, 2H), ppm.

### Step 2A. Preparation of (4S)-4-phenyl-3-[(5S)-5-(4-fluorophenyl)-5-hydroxypentanoyl]-1,3-oxazolidin-2-one (A2 R⁶ =phenyl)

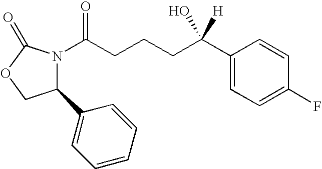

(4S)-4-Phenyl-3-[5-(4-fluorophenyl)-5-oxopentanoyl]-1,3-oxazolidin-2-one (A1 R⁶=phenyl) (28.43 g, 80.0 mmol) was dissolved in dichloromethane (160.0 mL; 0.5 M). The mixture was cooled to −10° C. (ice/brine bath), stirred for 10 min and charged with 1.0 M (R)-1-methyl-3,3-diphenyltetrahydro-3H-pyrrolo[1,2-c][1,3,2]oxazaborole in toluene (4.0 mL, 4.0 mmol), followed by dropwise addition of borane-methyl sulfide complex (7.80 mL, 6.26 g, 82.4 mmol). The addition rate was adjusted in order to keep the temperature at −8° C. The reaction temperature was maintained between −5 and −8° C. with stirring for 3.0 h. The reaction was quenched by slow addition of methanol (16.3 mL, 402.4 mmol), 6% aqueous hydrogen peroxide (68.2 mL, 120.0 mmol) and 1.0 M aqueous sulfuric acid (48.0 mL, 48 mmol) respectively, with ice-bath cooling. The cooling bath was then removed and the reaction was stirred at room temperature. After stirring at room temperature for 45 min, the mixture was poured into a separatory funnel, the organic layer was separated and the aqueous layer was extracted with dichloromethane (200 mL). The first organic layer was washed with water (125 mL) and brine (125 mL). The aqueous layers were backed extracted with the second organic layer. The combined organic layers were dried over sodium sulfate, filtered through Celite®, and concentrated to afford 31.9 g of a clear viscous film as crude product. This film was dissolved in 60 ml toluene at 50° C., cooled to room temperature, and crystallized over 12 h at −15° C. The white crystalline solid was filtered, transferred and washed with cold toluene (100 mL), air dried and vacuum dried to afford 24.45 g of a white solid. NMR analysis indicated the product to contain 6% toluene. The solid was again dissolved in toluene (50 mL) at 50° C. and hexane (50 mL) was added. The solution was cooled to room temperature with stirring and then stirred in an ice bath for 1 h. The white solid was filtered, transferred and washed with hexane (200 mL), air dried and vacuum dried to constant weight to afford (4S)-4-phenyl-3-[(55)-5-(4-fluorophenyl)-5-hydroxypentanoyl]-1,3-oxazolidin-2-one (22.56 g, 79% yield) as a white crystalline solid; m.p. 39.7° C.; R_{f} 0.21 (2:3 ethyl acetate-hexane); HPLC R_{T} 6.09 min; HPLC purity 96.5%; ¹H NMR (300 MHz, CDCl₃) δ 7.15-7.42 (m, 7H), 7.00 (t, J=8.8 Hz, 2H), 5.40 (dd, J=3.7, 8.7 Hz, 1H), 4.68 (t, J=8.8 Hz, 1H), 4.59-4.66 (m, 1H), 4.27 (dd, J=3.7, 9.1 Hz, 1H), 2.93 (dt, J=1.1, 6.2 Hz, 2H), 1.58-1.80 (m, 4H) ppm.

### Step 5A. Preparation of 3-[2-[(2-Benzyloxy-4-bromo-phenyl)-phenylamino-methyl]-5-(4-fluoro-phenyl)-5-hydroxy-pentanoyl]-4-phenyl-oxazolidin-2-one(Diphenyl)

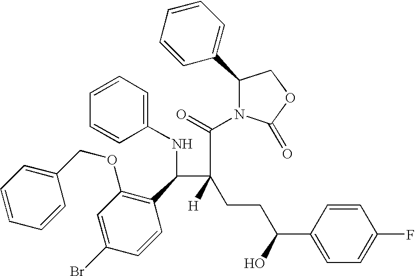

(4S)-4-phenyl-3-[(5S)-5-(4-fluorophenyl)-5-hydroxypentanoyl]-1,3-oxazolidin-2-one (A2phenyl) (21.4 g, 58.6 mmol) was dissolved in anhydrous dichloromethane (100 mL, 0.6 M) and cooled to −45° C. N-ethyldiisopropylamine (21.9 mL, 16.3 g, 125.8 mmol) was added slowly, followed by chlorotrimethylsilane (8.0 mL, 6.83 g, 62.9 mmol). The reaction was stirred for 1 h and the temperature was maintained between −20 and −30° C. Titanium tetrachloride (6.90 mL, 11.9 g, 62.9 mmol) was added drop-wise over 20 min to afford a deep reddish purple solution. The temperature was kept between −30 and −35° C. and stirring was continued for 45 min. The mixture was then cooled to −45° C. and a solution of N-{(1E)-[2-(benzyloxy)-4-bromophenyl]methylene}-N-phenylamine (B3) (37.3 g, 101.8 mmol) in dichloromethane (100 mL, 1.0 M) was added drop-wise over 30 min. The reaction temperature was maintained between −40° C. and −45° C. during addition. The mixture was stirred for 1.5 h between −40° C. and −45° C. An aliquot was removed for analysis by TLC and HPLC. The reaction was quenched by slow addition of glacial acetic acid (13.7 mL, 14.4 g, 240.0 mmol) over 10 min, followed by addition of cold (10° C.) 15% aqueous dl-tartaric acid solution (240.0 mL, 36.0 g, 240.0 mmol). The reaction mixture was warmed to −5° C. and was further allowed to warm up to room temperature after tartaric acid addition was completed. The mixture was stirred at room temperature over the next 1.5 h, diluted with dichloromethane (200 mL), poured into a separatory funnel and the layers were separated. The organic layer was washed with dilute brine solution (9:1 water/brine, 250 mL), then brine (100 mL). The aqueous layer was re-extracted sequentially with 1:1 ethyl acetate-hexane (200 mL, 150 mL). The combined organic layers were dried over Na₂SO₄ and concentrated to afford 59.4 g of an orange-red viscous oil. The crude product was dissolved in methanol (250 mL) and stored at −15° C. for 12 h. The resulting slurry was filtered to afford a white solid (27.7g), suspended in methanol (150 mL) at 55° C., cooled in an ice-bath with stirring for 30 min to afford a white solid, filtered, transferred and washed with cold methanol (150 mL), air-dried and high-vacuum dried to afford 3-[2-[(2-Benzyloxy-4-bromo-phenyl)-phenylaminomethyl]-5-(4-fluoro-phenyl)-5-hydroxy-pentanoyl]-4-phenyl-oxazolidin-2-one D1phenyl (22.1 g, 51% yield) as a white powder; R_{f} 0.32 (1:1 ethyl acetate-Hexane); HPLC R_{T} 10.24 min; HPLC purity ≦99%; ¹H NMR (300 MHz, CDCl₃) δ 7.51 (dd, J=1.6, 8.3 Hz, 2H), 6.67-7.40 (m, 17H), 6.59 (tt, J=1.0, 7.4 Hz, 1H), 6.39 (dd, J=1.1, 8.6 Hz, 2H), 5.31-5.42 (m. 2H), 5.04-5.25 (m, 2H), 4.92 (dd, J=6.0, 9.5 Hz, 1H), 4.80 (dd, J=6.9, 13.3 Hz, 1H), 4.66 (t, J=8.6 Hz, 1H), 4.45-4.54 (m, 1H), 4.13 (dd, J=3.5, 8.8 Hz, 1H), 1.89 (d, J=3.4 Hz, 2H), 1.58-1.84 (m, 3H) ppm.

### Step 6A. Preparation of (3R,4S)-4-[2-(benzyloxy)-4-bromophenyl]-3-[(3S)-3-(4-fluorophenyl)-3-hydroxypropyl]-1-phenylazetidin-2-one (D2)

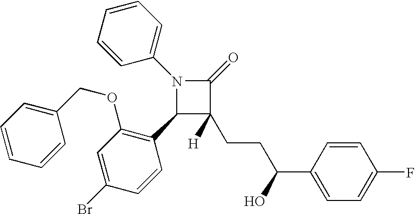

A 100 mL flask was charged with 3-[2-[(2-Benzyloxy-4-bromo-phenyl)-phenylamino-methyl]-5-(4-fluoro-phenyl)-5-hydroxy-pentanoyl]-4-phenyl-oxazolidin-2-one (D1phenyl) (1.45 g, 2.00 mmol) in anhydrous tert-butyl methyl ether (10 mL, 0.2 M) and N,O-bistrimethylsilylacetamide (1.0 mL, 4.00 mmol) was added. The clear solution was heated at reflux for 2 h with stirring. The heating bath was removed and a catalytic amount of tetrabutylammonium fluoride hydrate (0.050 g, 0.20 mmol) was added to afford a color change from colorless to pale yellow. Additional N,O-bistrimethylsilylacetamide (0.5 mL, 2.00 mmol) was added and the solution was stirred at room temperature for 16 h. The reaction was then cooled on ice and glacial acetic acid (0.01 mL, 0.20 mmol) was added, followed by 1.0 N aqueous hydrochloric acid (3.5 mL), which was added drop-wise to avoid an exotherm (decomposition of the N,O-bistrimethylsilylacetamide with aqueous acid can be reactive). The bright yellow biphasic mixture was stirred for 0.5 h, poured into a separatory funnel, diluted with 1:1 ethyl acetate-hexane (50 mL) and water (50 mL), agitated, the layers were separated and the organic layer was washed with water (50 mL) and brine (50 mL). The two aqueous layers were back-extracted sequentially with two portions of 1:1 ethyl acetate-hexane (2×30 mL) and the combined organic layers were dried over sodium sulfate and concentrated to afford 1.60 g yellow oil. The product was purified by column chromatography (ethyl acetate/hexane gradient 1:9 to 1:1) to afford (3R,4S)-4-[2-(benzyloxy)-4-bromophenyl]-3-[(35)-3-(4-fluorophenyl)-3-hydroxypropyl]-1-phenylazetidin-2-one D2 (0.687 g, 61%) as a white solid (purity ≧99% by LC-MS, R_{f}=0.30 [2:1 hexane/ethyl acetate], M(—OH—): 542.4 m/z); ¹H NMR (300 MHz, CDCl₃) δ 7.41 (br s, 5H), 7.28-7.22 (m, 4H), 7.19-7.15 (m, 3H), 7.08-7.02 (m, 3H), 6.96 (t, J=8.7 Hz, 2H), 5.10 (dd, J=15.2, 11.2 Hz, 2H), 5.01 (d, J=2.4 Hz, 1H), 4.57-4.52 (m, 1H), 3.06-3.00 (m, 1H), 2.25 (d, J=3.8, 1H), 1.97-1.74 (m, 4H) ppm; [α]_{D}²³−12.3° (c 6.5, ethyl acetate).

An alternative procedure used to crystallize D2 was as follows: The diastereomer ratio of D1 starting material was 79:21 [trans(total):cis(total)]. The crude D2 after work-up of the cyclization reaction, which totaled 135 g (Theory: 117 g of D2 diastereomers plus up to 37 g of cleaved benzyloxazolidinone) was heated in methanol (700 mL) to 65° C. Water (90 mL) was added dropwise to the stirred solution over 10 minutes. Seeds of diastereomerically pure D2 occasionally were added to the solution as it was cooled slowly to 47° C., held at 47° C. overnight, then finally cooled to room temperature over 5 hr. The solid was collected by filtration, then washed with ice-cold methanol/water (89:11) and dried under vacuum to give an off-white solid (D2, 54.0 g). No cis diastereomer could be detected by ¹H-NMR. The solid was heated to 50° C. in a mixture of methanol and isopropyl alcohol and charcoal was added. The solution was filtered and concentrated to dryness to give 43.9 g of white solid. This material was heated to 73° C. in isopropyl alcohol (228 mL) and a mixture of isopropyl alcohol/water (27:73, 104 mL) was added over 45 min. The solution was cooled to 65° C., seed crystals of diastereomerically pure D2 were added and the solution was allowed to cool slowly to room temperature. The solid was collected by filtration, washed with isopropyl alcohol/water (75:25, 80 mL) and dried under vacuum to give pure (3R,4S)-4-[2-(benzyloxy)-4-bromophenyl]-3-[(3S)-3-(4-fluorophenyl)-3-hydroxypropyl]-1-phenylazetidin-2-one (D2, 40.7 g, 44% yield from D1) as white needles, mp 113.9° C. The diastereomeric purity was determined to be 99.9% by chiral hplc analysis.

### Step 7. Preparation of (1S)-1,5-anhydro-2,3,4,6-tetra-O-benzyl-1-[4-(4,4,5,5-tetramethyl-1,3,2-dioxaborolan-2-yl)phenyl]-D-glucitol (C4-benzyl)

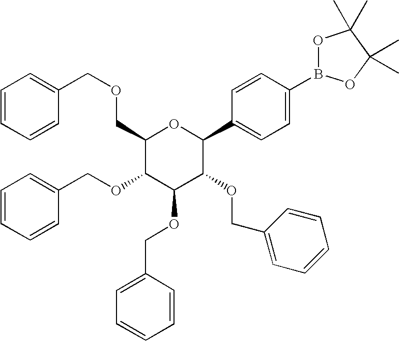

A reactor was charged with (1S)-1,5-anhydro-2,3,4,6-tetra-O-benzyl-1-(4-bromophenyl)-D-glucitol (30.0 kg, 44.1 mol), bis(pinacolato)diboron (14.6 kg, 57.5 mol) and potassium acetate (13.2 kg, 134.5 mol) and the solids were dissolved in dimethylsulfoxide (150 kg). Dichloro[1,1′-bis(diphenylphosphino)ferrocene]palladium(II) dichloromethane adduct (1.45 kg, 1.77 mol) was added as a slurry in dimethylsulfoxide (2×5 kg) and the reaction was degassed for 30 min. The reaction was sealed and heated to 87±3° C. for 2 h. The mixture was cooled to 15° C., poured into water (300 kg) and tert-butylmethylether (220 kg), agitated, filtered through Celite®, the layers were separated and the aqueous layer was back extracted with tert-butylmethylether (145 kg). The combined organic layers were washed with water (3×300 kg) and 25% (w/w) aqueous sodium chloride solution (200 kg), dried over sodium sulfate (3.5 kg) and filtered. The mixture was treated with charcoal (12 kg), heated to 40±5° C. for 20 min, cool to 20±5° C. for 20 min, filtered through Celite® and concentrate in vacuo. The residue was suspended in ethyl acetate (27 kg) and hexane (79 kg) was added portion-wise, silica gel was added (40 kg) and the mixture was filtered and eluted with 4:1 hexane-ethyl acetate until the product was eluted off. The compound rich eluent was concentrated in vacuo to afford (1S)-1,5-anhydro-2,3,4,6-tetra-O-benzyl-1-[4-(4,4,5,5-tetramethyl-1,3,2-dioxaborolan-2-yl)phenyl]-D-glucitol (C4-benzyl) (31.8 g, 99% yield) as a viscous oil; R_{f} 0.51 (1:4 ethyl acetate-hexane); ¹H NMR (300 MHz, CDCl₃) δ 7.84 (d, J=7.7 Hz, 2H), 7.50 (d, J=7.7 Hz, 2H), 7.38-7.19 (m, 18H), 6.97-6.94 (m, 2H), 4.93 (dd, J=17.8, 11.1 Hz, 2H), 4.89 (d, J=10.6 Hz, 1H), 4.66 (d, J=10.6 Hz, 1H), 4.63 (dd, J=28.1, 12.3 Hz, 2H), 4.34 (d, J=10.4 Hz, 1H), 4.28 (d, J=9.4 Hz, 1H), 3.85-3.73 (m, 5H), 3.64-3.59 (m, 1H), 3.56-3.50 (m, 1H), 1.38 (s, 12H) ppm.

### Step 8. Preparation of (1S)-1,5-anhydro-1-[4-(4,4,5,5-tetramethyl-1,3,2-dioxaborolan-2-yl)phenyl]-D-glucitol (C4-hydroxyl)

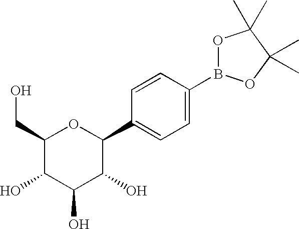

A reactor was charged with (1S)-1,5-anhydro-2,3,4,6-tetra-O-benzyl-1-[4-(4,4,5,5-tetramethyl-1,3,2-dioxaborolan-2-yl)phenyl]-D-glucitol (31.8 kg, 43.8 mol) and was dissolved in tetrahydrofuran (90 kg). 10% Palladium on carbon (50% wet, 3.5 kg) was added as a slurry in tetrahydrofuran (8 kg) and another portion of tetrahydrofuran (2 kg) was used to transfer residual catalyst. The vessel was vacuum/nitrogen purged, pressurized with hydrogen to 30±5 psi and vented three times before finally pressurizing to 50 psi. The mixture was heated at 30±5° C. for 24 h (maintaining a pressure of 50 psi as needed), cooled to 20±5° C. and then pressurized with nitrogen to 30±5 psi and vented (five cycles). The solution was filtered and the cake was washed with tetrahydrofuran (75 kg) to afford a solution of (1S)-1,5-anhydro-1-[4-(4,4,5,5-tetramethyl-1,3,2-dioxaborolan-2-yl)phenyl]-D-glucitol (C4-hydroxyl) which was used as is for the next reaction. ¹H NMR (300 MHz, CDCl₃) δ 7.78 (d, J=7.5 Hz, 2H), 7.33 (d, J=7.5 Hz, 2H), 4.02 (d, J=8.6 Hz, 1H), 3.78-3.69 (m, 2H), 3.57-3.46 (m, 2H), 3.38-3.32 (m, 1H), 3.27-3.23 (m, 1H), 1.27 (s, 12H) 1.14 (br s, 4H) ppm.

### Step 9. Preparation of (1S)-2,3,4,6-tetra-O-acetyl-1,5-anhydro-1-[4-(4,4,5,5-tetramethyl-1,3,2-dioxaborolan-2-yl)phenyl]-D-glucitol (C4-acetyl)

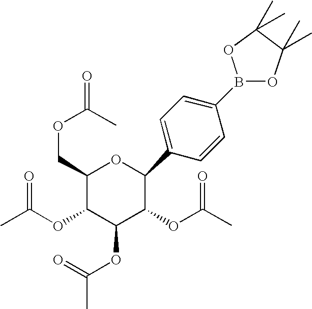

The solution of (1S)-1,5-anhydro-1-[4-(4,4,5,5-tetramethyl-1,3,2-dioxaborolan-2-yl)phenyl]-D-glucitol in tetrahydrofuran (175 kg) from step 8 was charged with pyridine (25.4 kg) and acetic anhydride (32.8 kg). The reaction was agitated at 50±5° C. for 12 h then cooled to to 15° C., poured into tert-butylmethylether (145 kg), and the pH of the mixture was adjusted with 1.0 M aqueous hydrochloric acid (160 kg) to a pH of about 4. The solution was agitated for 5 min, the layers were separated and the aqueous layer was back extracted with tert-butylmethylether (90 kg). The combined organic layers were washed with water (2×190 kg) and 25% (w/w) aqueous sodium chloride solution (200 kg), dried over sodium sulfate (3.5 kg), filtered, and concentrate in vacuo. The residue was purified by crystallization from isopropanol (135 kg) to afford (1)-2,3,4,6-tetra-O-acetyl-1,5-anhydro-1-[4-(4,4,5,5-tetramethyl-1,3,2-dioxaborolan-2-yl)phenyl]-D-glucitol (C4-acetyl) (13.15 kg, 57.7% yield over two steps); ¹H NMR (300 MHz, CDCl₃) δ 7.76 (d, J =8.1 Hz, 2H), 7.33 (d, J=8.1 Hz, 2H), 5.31 (d, J=9.0 Hz, 1H), 5.2 (t, J=9.5 Hz, 1H), 5.1 (t, J=9.5 Hz, 1H), 4.40 (d, J=9.9 Hz, 1H), 4.30 (dd, J=5.1, 4.8 Hz, 1H), 4.15 (dd, J=2.4, 2.1 Hz, 1H), 3.86-3.80 (m, 1H), 2.08 (s, 3H), 2.06 (s, 3H), 1.99 (s, 3H), 1.79 (s, 3H), 1.34 (s, 12H) ppm.

### Step 10. Preparation of (1S)-2,3,4,6-tetra-O-acetyl-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E1)

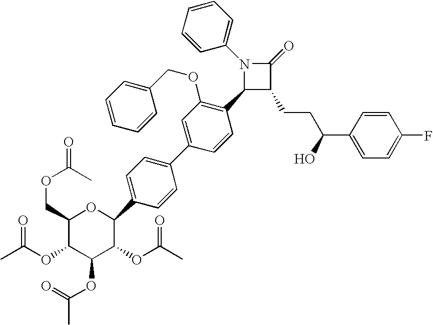

A 1-L three-necked flask was charged with (3R,4S)-4-[2-(benzyloxy)-4-bromophenyl]-3-[(3S)-3-(4-fluorophenyl)-3-hydroxypropyl]-1-phenylazetidin-2-one (48.6 g, 0.087 mol) and (1S)-2,3,4,6-tetra-O-acetyl-1,5-anhydro-1-[4-(4,4,5,5-tetramethyl-1,3,2-dioxaborolan-2-yl)phenyl]-D-glucitol (48.5 g, 0.091 mol) followed by addition of degassed toluene (174.0 mL, 0.5 M). The mixture was stirred at room temperature until the starting materials dissolved and then nitrogen gas was bubbled directly into the solution for 30 min to displace oxygen. Degassed 2.0 M aqueous potassium carbonate (87.0 mL, 0.174 mol) was added followed by addition of solid triphenylphosphine (2.274 g, 0.00868 mol) and palladium (II) chloride (0.772 g, 0.00435 mol). Under certain circumstances it has been found advantageous to employ a bicarbonate base and quaternary ammonium phase transfer catalyst in place of the potassium carbonate, by which means the yield may be increased by up to ten percent. Nitrogen gas was bubbled directly into the solution for an additional 30 min to displace oxygen. The solution turned a rusty color and the mixture was heated to 90° C. (heating turns the solution a pale dark green color and upon reaching 80° C. the reaction turns black). The reaction was stirred for 3 h at 90° C., cooled to room temperature, poured into water (750 mL), extracted with 1:1 ethyl acetate-heptane (750 mL) and washed with brine (500 mL). The aqueous layers were back-extracted sequentially with 1:1 ethyl acetate-heptane (750 mL) and the organic layers were combined and concentrated. The residue was diluted with 30% ethyl acetate-hexane (800 mL), charged with silica gel (100 mL) and purified by pad filtration (1200 mL silica gel, 30% ethyl acetate-hexane (2000 mL), 33% ethyl acetate-hexane (2000 mL), 35% ethyl acetate-hexane (1000 mL) and 38% ethyl acetate-hexane (1000 mL) to remove impurities then 40% ethyl acetate-hexane (4000 mL) and 45% ethyl acetate-hexane (6500 mL)) to afford (1S)-2,3,4,6-tetra-O-acetyl-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E1) (60.0 g, 78% yield) as a light yellow foam; R_{f} 0.20 (1:1 ethyl acetate-hexane); HPLC purity 98.3 A %. ¹H NMR (300 MHz, CDCl₃) δ 7.52 (d, J=8.3 Hz, 2H), 7.46-7.34 (m, 6H), 7.32-7.02 (m, 10H), 6.95 (t, J=8.8 Hz, 2H), 6.93-6.87 (m, 1H), 5.39-5.16 (m, 4H), 5.12 (d, J=2.5 Hz, 1H), 4.57 (t, J=5.8 Hz, 1H), 4.46 (d, J=9.9 Hz, 1H), 4.31 (dd, J=12.4, 4.7 Hz, 1H), 4.18 (dd, J=12.4, 2.1 Hz, 1H), 3.87 (ddd, J=9.9, 4.7, 2.2 Hz, 1H), 3.13-3.07 (m, 1H), 2.09 (s, 3H), 2.07 (s, 3H), 2.02 (s, 3H), 1.92-1.82 (m, 4H), 1.82 (s, 3H) ppm.

### Step 11. Preparation of (1S)-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E2)

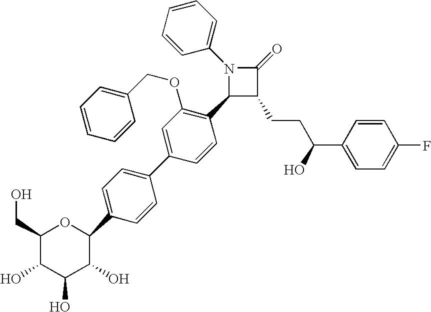

A 500-mL three-necked flask was charged with (IS)-2,3,4,6-tetra-O-acetyl-1,5-anhydro-1-(3′-(benzyloxy)-4′-{(2S,3R)-3-[(3S)-3 -(4-fluorophenyl)-3-hydroxypropyl]-4-oxo-1-phenylazetidin-2-yl}biphenyl-4-yl)-D-glucitol (60.0 g, 0.0676 mol) in methanol (220 mL, 0.31 M) and the mixture was heated to 40° C. 28% Aqueous ammonium hydroxide (110 mL, 1.87 mol) was added drop-wise via addition funnel at 40° C. over 45 min and then the mixture was heated for 3 h at 40° C. The reaction was concentrated in vacuo to remove the ammonia, treated with decolorizing charcoal (3.0 g) in methanol, heated, cooled, filtered through Celite® and rinsed with methanol. The solution was concentrated in vacuo to afford (1S)-1,5-anhydro-1-(3′-(benzyloxy)-4′-{(2S,3R)-3 -[(3S)-3-(4-fluorophenyl)-3-hydroxypropyl]-4-oxo-1-phenylazetidin-2-yl}biphenyl-4-yl)-D-glucitol (E2) (53.9 g, 111% due to water and ammonium acetate) as an off-white foam; R_{f} 0.21 (1:19 methanol-ethyl acetate with 1% acetic acid); HPLC purity 95.8 A %. ¹H NMR (300 MHz, CD₃OD) δ 7.61-7.47 (m, 4H), 7.42-7.29 (m, 6H), 7.25-7.19 (m, 7H), 7.15-7.10 (m, 1H), 7.05-6.88 (m, 3H), 5.24 (d, J=11.9 Hz, 1H), 5.17 (d, J=11.9 Hz, 1H), 5.12 (d, J=2.4 Hz, 1H), 4.54-4.50 (m, 1H), 4.17 (d, J=9.2 Hz, 1H), 3.92-3.87 (m, 1H), 3.75-3.69 (m, 1H), 3.54-3.36 (m, 4H), 3.15-3.10 (m, 1H), 1.92-1.82 (m, 4H) ppm.

### Step 11A. Alternate Preparation of (1S)-1,5-anhydro-1-(3′-(benzyloxy)-4′-{(2S,3R)-3 -[(3S)-3-(4-fluorophenyl)-3-hydroxypropyl]-4-oxo-1-phenylazetidin-2-yl}biphenyl-4-yl)-D-glucitol (E2)

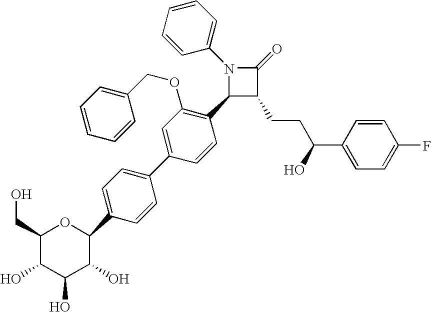

(1S)-2,3,4,6-Tetra-O-acetyl-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E1) (0.23 g, 0.25 mmol) and anhydrous potassium fluoride (0.06 g, 1.00 mmol) were dissolved in methanol (2 mL). The mixture was heated to 40° C. and stirred for 28 hours. After that time the reaction was determined complete by LCMS and poured into water (2 mL). Ethyl acetate (4 mL) was added and the product was extracted into the organic layer. The aqueous phase was once again extracted with ethyl acetate (4 mL), the organic layers were combine, dried with sodium sulfate and concentrated to a white foam. The crude product (1S)-1,5-anhydro-1-(3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)-D-glucitol (E2) (0.179 mg, 0.25 mmol, 100% yield) was determined to be 100% pure by LMCS; ¹H NMR (CDCl₃/300 MHz) 7.45 (q, 4H, J=8.1 Hz), 7.37 (m, 5H), 7.24 (m, 5H), 7.03 (m, 2H), 6.97 (m, 4H), 5.35 (m, 2H), 5.14 (d, 1H, J=2.1 Hz), 4.53 (m, 1H), 4.19 (d, 1 H, J=9.3 Hz), 3.87 (m, 1H), 3.73 (m, 1H), 3.42 (m, 2H), 3.17 (m, 1H), 1.88 (m, 4H) ppm.

### Step 12. Preparation of (1S)-1,5-anhydro-1-(4′-{(2S,3R)-3-[(3S)-3-(4-fluorophenyl)-3-hydroxypropyl]-4-oxo-1-phenylazetidin-2-yl}-3′-hydroxybiphenyl-4-yl)-D-glucitol (ADG)

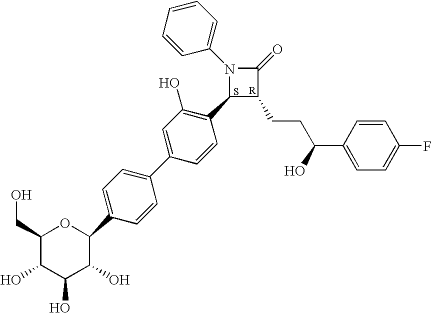

A 400-mL hydrogenation pressure flask was charged with crude (1S)-1,5-anhydro-1-(3′-(benzyloxy)-4′-{(2S,3R)-3-[(3s)-3-(4-fluorophenyl)-3-hydroxypropyl]-4-oxo-1-phenylazetidin-2-yl}biphenyl-4-yl)-D-glucitol (theoretical 67.6 mmol) in ethanol (180 mL). The 10% palladium on carbon (19.2 g, 0.0051 mol) was added as a solid, the flask was sealed with a rubber septum and the black solution was stirred vigorously. Hydrogen gas was then bubbled directly into the solution via a long syringe needle with the exhaust bubbling out through a large beaker of water. After 6 h of bubbling at room temperature the reaction was complete and the solution was purged with nitrogen gas for 30 min. The mixture was filtered through Celite® under a blanket of nitrogen gas, washed with 200-proof ethanol (400 mL), concentrated and then filtered through a 0.2 micron filter to remove particulate material. The compound was purified by reverse-phase HPLC (Dynamax compression module, Polaris 10 C18-A 10μ 250×41.4 mm column, batch 219504, isocratic 49% methanol-water, flow rate: 80 mL/min) to afford (1S)-1,5-anhydro-1-(4′-{(2S,3R)-3-[(3S)-3-(4-fluorophenyl)-3-hydroxypropyl]-4-oxo-1-phenylazetidin-2-yl}-3′-hydroxybiphenyl-4-yl)-D-glucitol (ADG) (28.4 g, 67% yield over two steps) as an off-white amorphous solid; m.p. 152-160° C.; HPLC purity 94.0 A %; ¹H NMR (300 MHz, CD₃OD) δ 7.54 (d, J=8.5 Hz, 2H), 7.47 (d, J=8.5 Hz, 2H), 7.35-7.09 (m, 8H), 7.05-6.97 (m, 4H), 5.14 (d, J=2.3 Hz, 1H), 4.63-4.59 (m, 1H), 4.17 (d, J=9.5 Hz, 1H), 3.90 (dd, J=11.8, 1.6 Hz, 1H), 3.71 (dd,J=11.8, 4.9 Hz, 1H), 3.53-3.36 (m, 4H), 3.19-3.13 (m, 1H), 2.05-1.88 (m, 4H) ppm; [α]_{D}²³+1.7° (c 8.7, methanol).

### Step 7A. Preparation of 2,3,4,6-tetra-O-acetyl-α-D-glucopyranosyl bromide (C2).

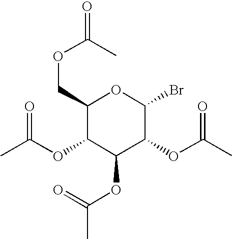

33% Hydrogen bromide in acetic acid (250 mL, 1.02 mol) was added dropwise to neat β-D-glucose pentaacetate (C1) (98.4 g, 0.25 mol) powder in a 2-L flask over 10 min at room temperature to afford a yellow solution. The mixture was stirred for 1 h at room temperature. The solvent was removed by azeotropic distillation in vacuo with toluene (3×100 mL) followed by high vacuum to afford 2,3,4,6-tetra-O-acetyl-α-D-glucopyranosyl bromide (C2) (quantitative) as a pale yellow waxy solid; R_{f} 0.49 (1:1 ethyl acetate-hexane); NMR purity >99 A %. ¹H NMR (CDCl₃) δ 6.62 (d, J=4.2 Hz, 1H), 5.56 (t, J=9.9 Hz, 1H), 5.17 (t, J=9.6 Hz, 1H), 4.84 (dd, J=9.9, 4.2 Hz, 1H), 4.36-4.27 (m, 2H), 4.16-4.11 (m, 1H), 2.11 (s, 3H), 2.10 (s, 3H), 2.06 (s, 3H), 2.04 (s, 3H).

### Step 8A. Preparation of (1S)-2,3,4,6-tetra-O-acetyl-1,5-anhydro-1-(4-bromophenyl)-D-glucitol (C3)

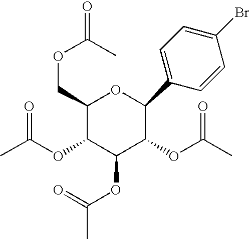

1,4-Dibromobenzene (713.4 g, 3.02 mol) was dissolved in anhydrous ether (1700 mL, 1.78 M). This solution was transferred portion-wise to a vapor equilibrating addition funnel (250 mL). A bulk portion (50 mL) of this solution followed by 1,2-dibromoethane (500 μL) was added to magnesium turnings (74.1 g, 3.05 mol) covered with anhydrous ether (300 mL). Within 2 min, the reaction became cloudy and solvent began to reflux. The dibromobenzene solution was added at such a rate to maintain a steady reflux and was added over 60 min. After approximately 3 min, the solution had developed a pale green color which proceeded to darken to a murky brown as the addition continued. After complete addition, the dark brown solution was diluted with anhydrous ether (200 mL) and stirred at room temperature for 1 h. The reaction was cooled to 0° C. with an ice bath. 2,3,4,6-Tetra-O-acetyl-α-D-glucopyranosyl bromide (C2) (103.6 g, 0.252 mol) dissolved in anhydrous ether (1000 mL) was added to the 4-bromophenylmagnesium bromide solution over 60 min with vigorous stirring. After complete addition, the solution was warmed to room temperature and stirred for 72 h. The reaction was cooled to 0° C. in an ice bath and carefully quenched with a solution of 10% acetic acid-water (1500 mL, 2.62 mol). The bulk of the aqueous layer was separated and the remaining mixture was filtered through Celite® to remove a greenish emulsion. The organic phase was extracted with 10% acetic acid solution (8×350 mL) keeping the individual extracts separate. Six of the eight fractions were combined with the original aqueous layer and evaporated in vacuo yielding a solid residue.

The residue isolated from the Grignard addition was dissolved in pyridine (2000 mL) and the mixture was cooled to 0° C. in an ice bath. N,N-Dimethylaminopyridine (0.8 g) was added followed by acetic anhydride (1000 mL, 10.58 mol). The reaction was stirred for 30 min at 0° C. then warmed to room temperature and stirred for 17 h. The reaction became very viscous with a large quantity of off-white precipitate. The reaction was divided into two approximately equal portions and individually diluted with diethyl ether (1 L). The solutions were filtered through paper in a Büchner funnel with suction washing the solid with additional diethyl ether and the bulk of the diethyl ether removed in vacuo. The residual pyridine solution was treated again with N,N-dimethylaminopyridine and acetic anhydride (200 mL, 2.11 mol) at 0° C. The reaction was left stirring at 0° C. for 1 h then warmed to room temperature for 17 h. The reaction was concentrated in vacuo to a final volumne of approximately 150 mL. The residue was dissolved in diethyl ether (500 mL) and washed with 3.0 N aqueous hydrochloric acid (100 mL) until the washes remained a pH <1. The ether solution was washed with water (100 mL), saturated aqueous sodium carbonate (150 mL), saturated aqueous sodium bicarbonate (2×150 mL), and water (100 mL) then dried over sodium sulfate, filtered and concentrated in vacuo yielding a light tan solid (93.9 g). Purification by pad filtration (470 g silica gel, loaded as a silica gel (100 g) slurry in 10:1 dichloromethane-10% ethyl acetate-hexane followed by eluting with 3.5 L 25% to 1.5 L 33% ethyl acetate-hexane) afforded (18)-2,3,4,6-tetra-O-acetyl-1,5-anhydro-1-(4-bromophenyl)-D-glucitol (C3) (66.4 g, 54% yield) as a white waxy solid. This material was recrystallized from isopropanol (266 mL) to recover a first crop of white solid (59.4 g, 40.6% yield, NMR Purity 84 A %) and a second crop (2.08 g, NMR Purity 60 A %); m.p. 131±0.8° C.; R_{f} 0.43 (1:1 ethyl acetate-hexane); ¹H NMR (300 MHz, CDCl₃) δ 7.47 (d, J =8.4 Hz, 2H), 7.31 (d, J=8.7, 2H), 5.31 (d, J=9.3 Hz, 1H), 5.21 (t, J=9.9 Hz, 1H), 5.09 (t, J=9.6 Hz, 1H), 4.37 (d, J=9.9 Hz, 1H), 4.12-4.33 (m, 2H), 3.83 (m, 1H), 2.09 (s, 3H), 2.06 (s, 3H), 2.00 (s, 3H), 1.83 (s, 3H) ppm.

### Step 9A. Preparation of (1S)-2,3,4,6-tetra-O-acetyl-1,5-anhydro-1-[4-(4,4,5,5-tetramethyl-1,3,2-dioxaborolan-2-yl)phenyl]-D-glucitol (C4-acetyl)

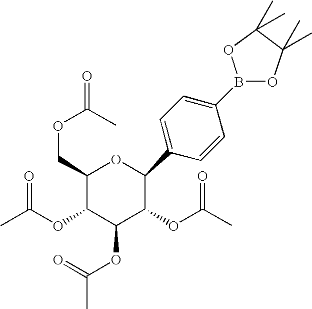

A 1-L three-necked flask was charged with (1S)-2,3,4,6-tetra-O-acetyl-1,5-anhydro-1-(4-bromophenyl)-D-glucitol (97.9 g, 0.20 mol) and dimethylsulfoxide (505 mL, 0.4 M). The reaction was degassed by bubbling nitrogen in the solution via a degassing stone. While vigorously bubbling, bis(pinacolato)diboron (61.0 g, 0.24 mol) and potassium acetate (59.9 g, 0.61 mol) were added as solids to the reaction followed by dichloro [1,1′-bis(diphenylphosphino)ferrocene]palladium(II) dichloromethane adduct (8.2 g, 0.01 mol). The reaction was degassed for an additional 30 min with nitrogen gas. Heating was applied over 40 min to achieve 88° C. and this temperature was maintained for 1.75 h. The reaction was cooled to room temperature, and poured into cold water (3300 mL) which was being mechanically stirred. The stirring was continued for 20 min after which time the mixture was filtered, and the solid was dried and collected. The resulting solid was dissolved in ethyl acetate (441 mL), diluted with hexane (906 mL), and charged with decolorizing charcoal (35.4 g), silica gel (35.4 g), and sodium sulfate (35.4 g). The slurry was heated while stirring for 15 min, cooled to room temperature and stirred for 15 min. The mixture was filtered through Celite® (150 mL) and washed with 33% ethyl acetate-hexane (2000 mL). The crude material was purified by crystallization from 1:6.4 ethyl acetate-hexane (740 mL, 6.9 mL/g theoretical yield) by first adding ethyl acetate (100 mL) followed by slow addition of hexane (640 mL) while stirring. The mixture was warmed to 55° C., stirred for 1 h and then slowly stirred to 32° C. over 4 h. The slurry was cooled to 10° C. for 1 h, filtered, washed with 5% ethyl acetate-hexane (800 mL) and dried to afford (1S)-2,3,4,6-tetra-O-acetyl-1,5-anhydro-1-[4-(4,4,5,5-tetramethyl-1,3,2-dioxaborolan-2-yl)phenyl]-D-glucitol (77.3 g, 72% yield in two crops) as a fine off-white powder; m.p. 135° C. (dec.); R_{f} 0.48 (1:1 ethyl acetate-hexane); HPLC purity >99%; NMR purity 95%. ¹H NMR (300 MHz, CDCl₃) δ 7.76 (d, J=8.1 Hz, 2H), 7.33 (d, J=8.1 Hz, 2H), 5.31 (d, J=9.0 Hz, 1H), 5.2 (t, J=9.5 Hz, 1H), 5.1 (t, J=9.5 Hz, 1H), 4.40 (d, J=9.9 Hz, 1H), 4.30 (dd, J=5.1, 4.8 Hz, 1H), 4.15 (dd, J=2.4, 2.1 Hz, 1H), 3.86-3.80 (m, 1H), 2.08 (s, 3H), 2.06 (s, 3H), 1.99 (s, 3H), 1.79 (s, 3H), 1.34 (s, 12H) ppm.

### Preparation of (3R,4S)-3-[(3S)-3-{[tert-butyl(dimethyl)silyl]oxy}-3-(4-fluorophenyl)propyl]-4-[2-{[tert-butyl(dimethyl)silyl]oxy}-4-(4,4,5,5-tetramethyl-1,3,2-dioxaborolan-2-yl)phenyl]-1-phenylazetidin-2-one

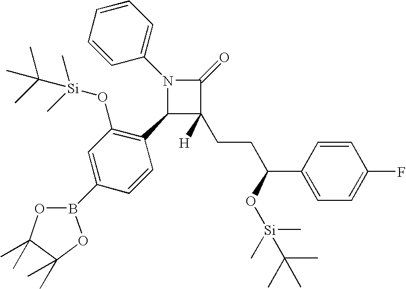

(3R,4S)-4-(4-Bromo-2-{[tert-butyl(dimethyl)silyl]oxy}phenyl)-3-[(3S)-3-{[tert-butyl(dimethyl)silyl]oxy}-3-(4-fluorophenyl)propyl]-1-phenylazetidin-2-one (0.42 g, 0.60 mmol) was dissolved in dioxane (15 mL) in a sealed tube. Bis(pinacolato)diboron (0.17 g, 0.66 mmol), potassium acetate (0.18g, 1.83 mmol), and dichloro[1,1′-bis(diphenylphosphino)ferrocene]palladium(II) dichloromethane adduct (14.6 mg, 0.018 mmol) were added and the reaction was degassed with argon and heated to 85° C. for 24 h. The mixture was cooled to room temperature diluted with 50 mL of 1:1 ethyl acetate-hexane, washed with 100 mL of 0.1 N hydrochloric acid and 2×100 mL of brine. The organic layers were collected, partially concentrated to half the volume, filtered through 10 g of silica gel, washed with 50 mL of ethyl acetate and concentrated in vacuo to afford (3R,4S)-3-[(3S)-3-{[tert-butyl(dimethyl)silyl]oxy}-3-(4-fluorophenyl)propyl]-4-[2-{[tert-butyl(dimethyl)silyl]oxy}-4-(4,4,5,5-tetramethyl-1,3,2-dioxaborolan-2-yl)phenyl]-1-phenylazetidin-2-one; ¹H NMR (300 MHz, CDCl₃) δ 7.35-7.18 (m, 9H), 7.02-6.96 (m, 1H), 6.95 (t, J=8.7 Hz, 2H), 5.11 (d, J=2.3 Hz, 1H), 4.63 (t, J=5.6 Hz, 1H), 3.06 (dt, J=7.4, 2.3 Hz, 1H), 1.96-1.79 (m, 4H), 1.31 (br s, 12H), 1.05 (s, 9H), 0.86 (s, 9H), 0.35 (s, 3H), 0.32 (s, 3H), 0.00 (s, 3H), -0.20 (s, 3H) ppm.

An alternative route to that of Step 9 for preparing C4-acetyl is shown in Scheme 6a, which is a particular embodiment of the route shown in Scheme 6.

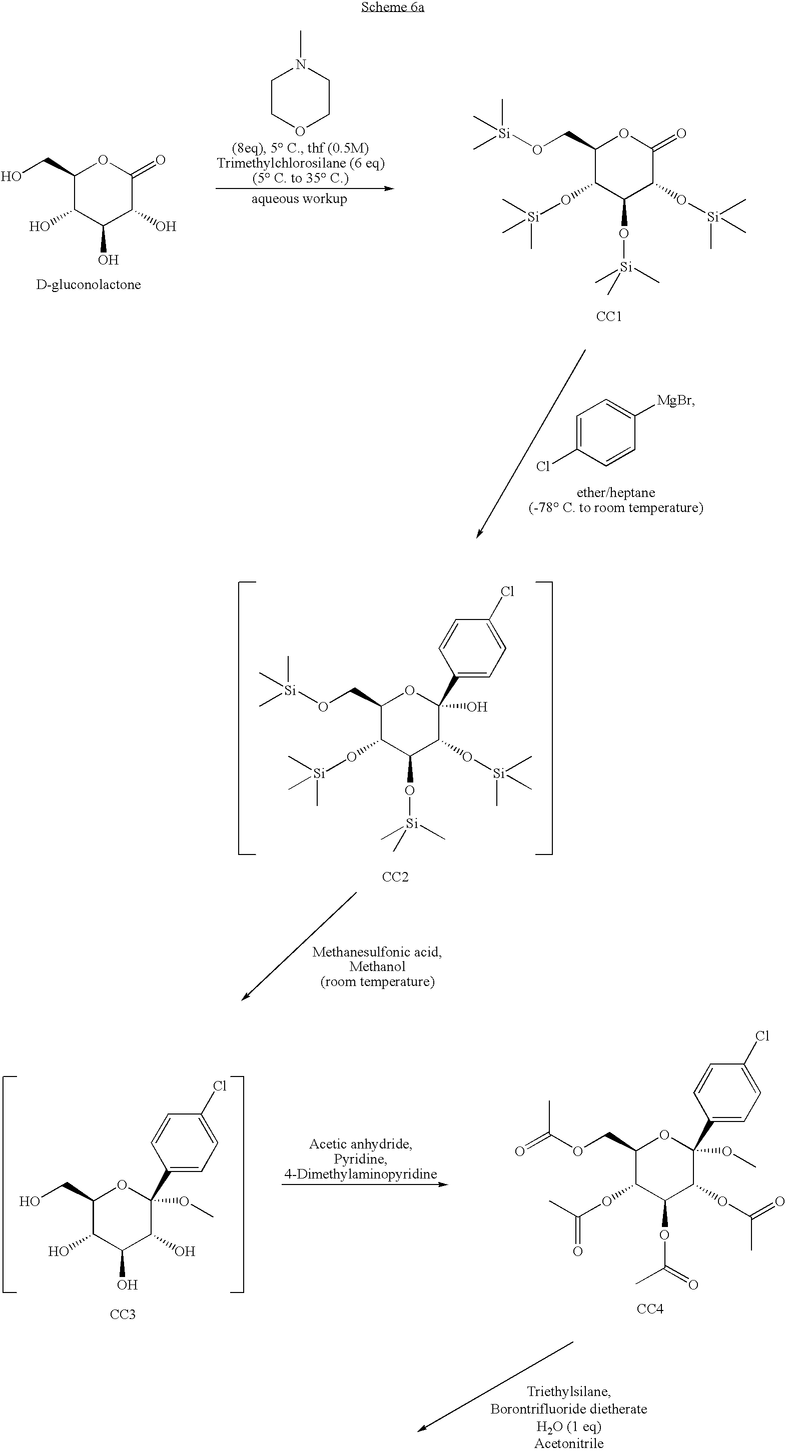

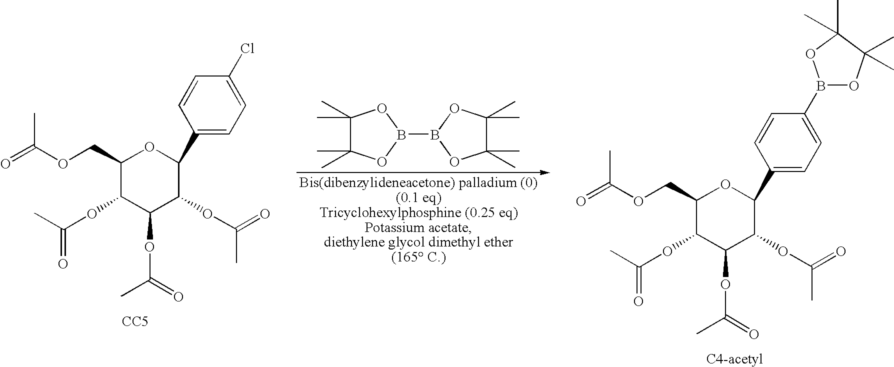

### Step 9B1. Preparation of 1-C-(4-chlorophenyl)-2,3,4,6-tetrakis-O-(trimethylsilyl)hexopyranose (CC2)

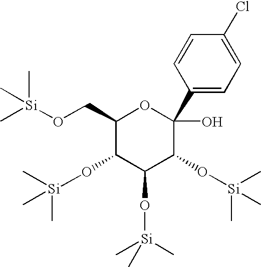

2,3,4,6-Tetrakis-O-(trimethylsilyl)-D-gluconic acid δ-lactone (CC1) was made from D-gluconolactone according to the methods described in US Patent Application Publication 2004/0137903 A1 and US Patent Application Publication 2004/0138439 A1.

2,3,4,6-tetrakis-O-(trimethylsilyl)-D-gluconic acid δ-lactone (CC1) (100.0 g, 0.214 mol) was dissolved in heptane (320.0 mL). The yellow solution was cooled to −78° C. in a dry ice/acetone bath and a 1.0 M solution of 4-chlorophenyl-magnesium bromide in diethyl ether (280 mL, 0.280 mol) was added dropwise over 20 minutes, controlling the rate of addition in order to maintain the temperature at or below −60° C. Stirring was continued for 1.0 h and the reaction temperature was maintained between −70 and −77° C. The cooling bath was then removed and the orange mixture was gradually warmed to room temperature over 1.5 h. The color of the reaction mixture changed from orange to yellow upon warming. After stirring at room temperature for 1.0 h the reaction was judged complete by LCMS analysis. The yellow reaction mixture was again cooled to −78° C. and quenched by slow addition of a saturated aqueous ammonium chloride solution (900 mL). The pale brown mixture was then warmed to room temperature over 30 min. After stirring for an additional 30 min at room temperature, the mixture was poured into a separatory funnel and the aqueous ammonium chloride layer was separated. The remaining organic layer was washed with brine (300 mL). The aqueous ammonium chloride layer was extracted with ethyl acetate (2×600 mL) and these extracts were used to consecutively back extract the first brine layer. Then, the original organic phase was washed with brine (200 mL) and back extracted, consecutively, with the two ethyl acetate layers. The organic layers were combined and concentrated to afford 133.9 g of 1-C-(4-chlorophenyl)-2,3,4,6-tetrakis-O-(trimethylsilyl)hexopyranose (CC2) as a pale brown oil; ¹H NMR (CDCl₃/300 MHz) 7.47 (d, J=8.4 Hz), 7.28 (d, J=8.1 Hz), 3.87 (m), 3.76 (d, J=3 Hz), 3.62 (t, J=8.7, 9.0 Hz), 3.42 (m), 0.20 (s), 0.18 (s), 0.08 (s), −0.30 (s) ppm.

### Step 9B2. Preparation of methyl 1-C-(4-chlorophenyl)hexopyranoside (CC3)

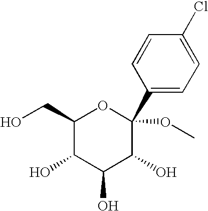

The crude product 1 -C-(4-chlorophenyl)-2,3,4,6-tetrakis-O-(trimethylsilyl)hexopyranose (CC2) (133.9 g) was dissolved in methanol (500 mL) at room temperature and methanesulfonic acid (69.6 mL, 1.07 mol) was added. Over the first 1.5 h to 2 h, the color of the reaction mixture changed gradually to dark purple and stirring was continued at room temperature for 24 h. The mixture was then cooled in an ice/water bath and triethylamine (287.2 mL) was added. The color of the mixture turned dark brown and was stirred with cooling using an ice/water bath for 5 min during which time the temperature fell to 18° C. The cooling bath was removed and stirring was continued at room temperature for 15 min. The reaction mixture was then concentrated to afford 283.9 g of a dark brown oil as the crude product methyl 1-C-(4-chlorophenyl)hexopyranoside (CC3); ¹H NMR (CDCl₃/300 MHz) 7.43(d, J=8.7 Hz), 7.20(d, J=8.7 Hz), 3.85(m), 3.55 (m) ppm.

### Step 9B3. Preparation of methyl-2,3,4,6-tetra-O-acetyl-1-C-(4-chloro-phenyl)-α-D-glucopyranose (CC4)

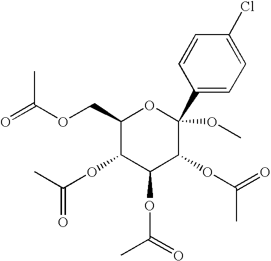

The crude product methyl I-C-(4-chlorophenyl)hexopyranoside (CC3) (283.9 g) was dissolved in pyridine (600 mL) and 4-dimethylaminopyridine (8.0 g, 0.066 mol) was added. The brown solution was cooled in an ice water bath and acetic anhydride (248.4 g, 2.43 mol) was added over 10 minutes in order to maintain the internal temperature at or below 11° C. Once the addition was complete, the cooling bath was removed and the dark orange-brown solution was stirred at room temperature for 30 min. The reaction was then quenched by addition of water (1000 mL) and stirred at room temperature. Extraction of the product was achieved by adding water (500 mL) and 20% ethyl acetate-heptane (1500 mL) and separating the phases. The organic layer was washed consecutively with 2.5 N HCl (1500 mL), water (1500 mL), and brine (1000 mL). The original aqueous layer was extracted with 20% ethyl acetate-heptane (1500 mL), which was then used to consecutively extract each of the previous aqueous washes. The organic layers were combined and silica gel (100 g) was added. The slurry was filtered over Celite™ (90 g) and washed with 20% ethyl acetate-heptane (2×500 mL). The yellow filtrate was concentrated to afford 84.8 g of an orange solid, which was then diluted in methanol (500 mL) and charged with charcoal (30 g). The slurry was stirred at 50° C. for 10 min, at room temperature for 30 min and was filtered through Celite® (90 g). The filter cake was washed with methanol (300 mL) and the filtrate was concentrated to afford 79.8 g of a yellow solid. The crude product was further purified by crystallization from 1:4 toluene-heptane (325 mL; 3.2 mL/g based on theoretical yield) using the following method. The crude product (79.8 g) was dissolved in toluene (65 mL), and stirred at 65° C. Heptane (260 mL) was added slowly over 5 min, maintaining the temperature at 65° C. The crystallization was seeded at 64° C. and the yellow solution was cooled to room temperature over 2 h, then stirred at room temperature for 14 h. The slurry was cooled in an ice water bath and stirred at 0° C. for 1 h. The white precipitate was filtered, washed with cold heptane (400 mL), air-dried and vacuum dried to afford methyl-2,3,4,6-tetra-O-acetyl-1-C-(4-chloro-phenyl)-α-D-glucopyranose CC4 (62.8 g, 62% yield from CC1) as a white crystalline solid; m.p. 161.1±0.15° C.; R_{f} 0.34 (40% ethyl acetate-hexane); ¹H NMR (CDCl₃/300 MHz) 7.39 (d, 2H, J=9.0 Hz), 7.33 (d, 2H, J=9.0 Hz), 5.60 (dd, 1H, J=9.6, 9.9 Hz), 5.23 (dd, 1H, J=9.6, 9.9 Hz), 4.94 (d, 1H, J=9.9 Hz), 4.37 (dd, 1H, J=5.0, 12.0 Hz), 4.23 (dd, 1H, J=2.3, 12.0 Hz), 4.05 (ddd, 1H, J=2.3, 5.0, 9.9 Hz), 3.12 (s, 3H), 2.12 (s, 3H), 2.06 (s, 3H), 1.96 (s, 3H), 1.96 (s, 3H) ppm. IR (solid) 1744.4, 13669, 1210.6, 1169.27, 1089.8, 1029.9, 965.5, 833.8 cm⁻¹.

### Step 9B4. Preparation of 2,3,4,6-tetra-O-acetyl-1-C-(4-chloro-phenyl)-β-D-glucopyranose (CC5)

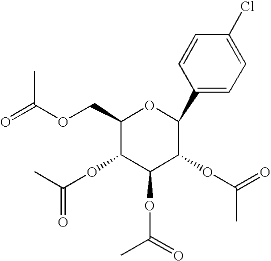

Methyl-2,3,4,6-tetra-O-acetyl-1-C-(4-chloro-phenyl)-α-D-glucopyranose (65.5 g, 0.144 mol) was dissolved in acetonitrile (800 mL) The yellow solution was cooled to −7.5° C. in an ice brine bath. Water (2.5 mL, 0.141 mol) was added, followed by triethylsilane (66.4 mL, 0.422 mol). Borontrifluoride dietherate (34.8 mL, 0.288 mol) was added dropwise over 5 min, and the color of the reaction changed to orangy/red. Stirring with cooling was continued for 30 min, then the cooling bath was removed and allowed to warm to room temperature over 45 min. Stirring was continued for 16 h and the reaction was monitored by ¹H NMR and determined to be incomplete. Triethylsilane (6.7 mL, 0.042 mol) and borontrifluoride dietherate (3.5 mL, 0.028 mol) were added and stirring was continued for another 7 h. When it was determined that the reaction had only proceeded to 95%, additional triethylsilane (10.0 mL, 0.062 mol) and borontrifluoride dietherate (5.3 mL, 0.042 mol) were added and stirring was continued for 15 h. An aqueous solution of saturated sodium bicarbonate (1000 mL) was added and the reaction mixture was stirred at room temperature for 60 min. The yellow suspension was transferred to a separatory funnel and the layers were separated. 1600 mL water and 1600 mL 40% ethyl acetate-heptane were then added to the acetonitrile layer. The mixture was agitated and the layers were again separated. The organic layer was washed consecutively with water (1600 mL) and saturated brine (1000 mL). The sodium bicarbonate layer, the two aqueous layers and the brine layer were extracted, consecutively, with 40% ethyl acetate-heptane (1600 mL). The organic phase was separated and combined with the first organic layer and concentrated to afford 64.2 g of 2,3,4,6-tetra-O-acetyl-1-C-(4-chloro-phenyl)-β-D-glucopyranose (CC5) as a crude pale yellow solid. The crude product was dissolved in hot ethanol (1000 mL). Charcoal (13 g) was added and the slurry was warmed for 5 min at 60° C. and filtered hot through Celite® (80 g). The filter cake was washed with hot ethanol (300 mL). The yellow filtrate was concentrated by heating at 60° C. to reduce the volume to 450 mL. The yellow solution was further stirred at 60° C. and was then allowed to cool slowly to room temperature. At 50° C. seeds were added and stirring was continued overnight. The resulting white crystals were filtered, washed with 300 mL of cold ethanol and dried to afford 2,3,4,6-tetra-O-acetyl-1-C-(4-chloro-phenyl)-β-D-glucopyranose (CC5) (35.7 g, 0.081 mol, 58% yield) as a fine white crystalline powder; m.p. 124.0±0.50° C. ° C.; R_{f} 0.35 (40% ethyl acetate-hexane); ¹H NMR (CDCl₃/300 MHz) 7.32 (d, 2H, J=7.0 Hz), 7.27 (d, 2H, J=7.0 Hz), 5.32 (dd, 1H, J=9.6, 9.6 Hz), 5.22 (dd, 1H, J=9.6, 9.6 Hz), 5.09 (dd, 1H, J=9.6, 9.6 Hz), 4.38 (d, 1H, J=9.6 Hz), 4.28 (dd, 1H, J=5.0, 12.6 Hz), 4.16 (dd, 1H, J=2.1, 12.6 Hz), 3.83 (ddd, 1H, J=2.1, 5.0, 9.6 Hz), 2.09 (s, 3H), 2.06 (s, 3H), 2.00 (s, 3H), 1.83 (s, 3H); IR (solid) 1739.7, 1371.1, 1213.4, 1116.4, 1090.4, 1030.6, 978.1, 919.4, 900.7, 831.2 cm⁻¹.

### Step 9B5. Preparation of (1S)-2,3,4,6-tetra-O-acetyl-1,5-anhydro-1-[4-(4,4,5,5-tetramethyl-1,3,2-dioxaborolan-2-yl)phenyl]-D-glucitol (C4-acetyl)

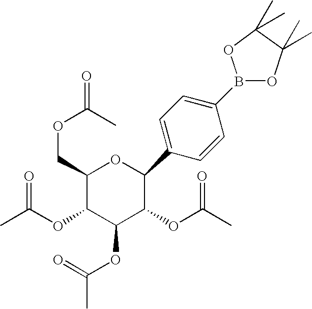

(1S)-2,3,4,6-Tetra-O-acetyl-1,5-anhydro-1-(4-chlorophenyl)-D-glucitol (CC5) (9.05 g, 0.020 mol), bis(pinacolato)diborane (5.7g, 0.023 mol), and potassium actetate (2.21g, 0.23 mol) were dissolved in anhydrous diglyme (50 mL). Argon was vigorously bubbled through the mixture while stirring. During this degassing the catalyst mixture was prepared by suspending bis(dibenzylideneacetone) palladium (0) (Pd(dba)₂) (1.15 g, 0.002 mol) and tricyclohexylphosphine (1.4 g, 0.023 mol) in anhydrous diethylene glycol dimethyl ether (40 mL). The catalyst mixture was stirred rapidly and vigorously degassed by bubbling argon. Both catalyst mixture and CC5 mixture were stirred and degassed for 1.25 h. After this time, the catalyst mixture was added to the CC5 mixture and degassing was continued for an additional 2 h. When degassing was complete the reaction was transferred to a 165° C. bath. The reaction turned from an orangy/tan color to grayish-brown upon heating. The reaction was allowed to stir at this temperature for approximately 18 h after which time the reaction was cool to room temperature. The crude reaction mixture was poured into ice water (300 mL) to precipitate the product. The flask was externally cooled to 0° C. and the mixture was stirred for 1.5 h. The resulting precipitate was collected by vacuum filtration and dissolved in ethyl acetate (40 mL). Hexane (80 mL) was then added and sodium sulfate (3.5 g) was added followed by silica gel (3.5 g) and charcoal (3.5 g). The mixture was heated to 40° C. and stirred for 15 minutes, filtered through Celite® (30 g) and washed with 33% ethyl acetate-hexane (225 mL). The organic filtrate was concentrated and 11.9 g of a yellow solid was collected. The crude material was crystallized by first dissolving in 50% ethyl acetate-hexane (44 mL) then adding hexane (40 mL). The crystallization was stirred over 16 hours, cooled to 0° C., filtered, washed with the mother liquor, and dried to afford (1S)-2,3,4,6-tetra-O-acetyl-1,5-anhydro-1-[4-(4,4,5,5-tetramethyl-1,3,2-dioxaborolan-2-yl)phenyl]-D-glucitol (C4-acetyl) (9.90 g, 92% yield from CC5) as a slight yellow crystalline solid; m.p. 140.9±0.50° C.; R_{f} 0.48 (50% ethyl acetate-hexane); ¹H NMR (CDCl₃/300 MHz) 7.75 (d, 2H, J=8.0 Hz), 7.32 (d, 2H, J=8.0 Hz), 5.31 (dd, 1H, J=9.3, 9.3 Hz), 5.20 (dd, 1H, J=9.3, 9.9 Hz), 5.10 (dd, 1H, J=9.3, 9.6 Hz), 4.39 (d, 1H, J=9.9 Hz), 4.27 (dd, 1H, J=5.0, 12.3 Hz), 4.13 (dd, 1H, J=2.1, 12.3 Hz), 3.81 (ddd, 1H, J=2.1, 5.0, 9.9 Hz), 2.06 (s, 3H), 2.03 (s, 3H), 1.97 (s, 3H), 1.78 (s, 3H), 1.25 (s, 12H) ppm. IR (solid) 1749, 1738, 1402, 1361, 1221, 1144, 1088, 1031, 916, 860, 834 cm⁻¹.

An alternative route to analogs of the compounds of formulas XIV and IV in Scheme 6 is shown in Scheme 7. According to this procedure, a compound of formula 101, obtained by the method of Braun and Cook [Org. Syn. 41, 79 (1961)] is reacted with an arylzinc according to the method of [J Org. Chem. 56, 1445-1453 (1991)] to obtain the phenylketone 102, which is then transformed to 104 in analogous fashion to that shown in Scheme 6.

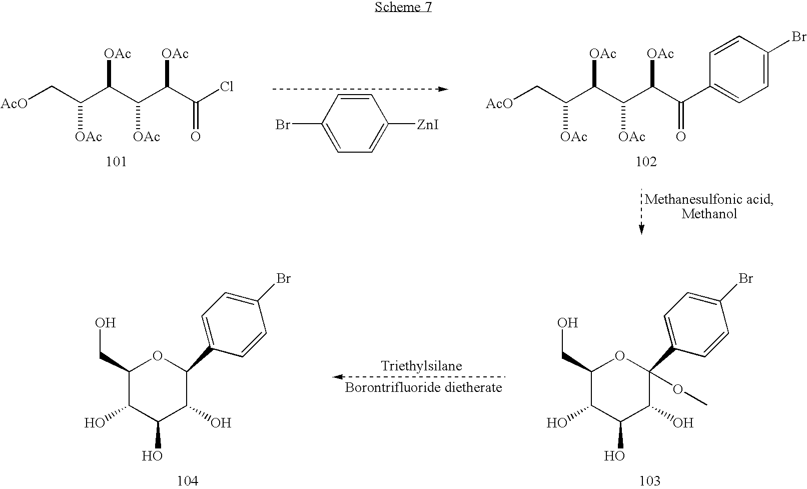

Another variation of the synthesis shown in Scheme 6 begins with gluconolactone and employs arylsilane 112 reaction with a chloroborane [see Kaufmann, Chem. Ber. 120, 853-854; 901-905 and Gross and Kaufmann, Chem. Ber. 120, 991-994 (1987)] to produce an unprotected sugar borane of formula 113 (C4-hydroxyl) as shown in Scheme 8 below:

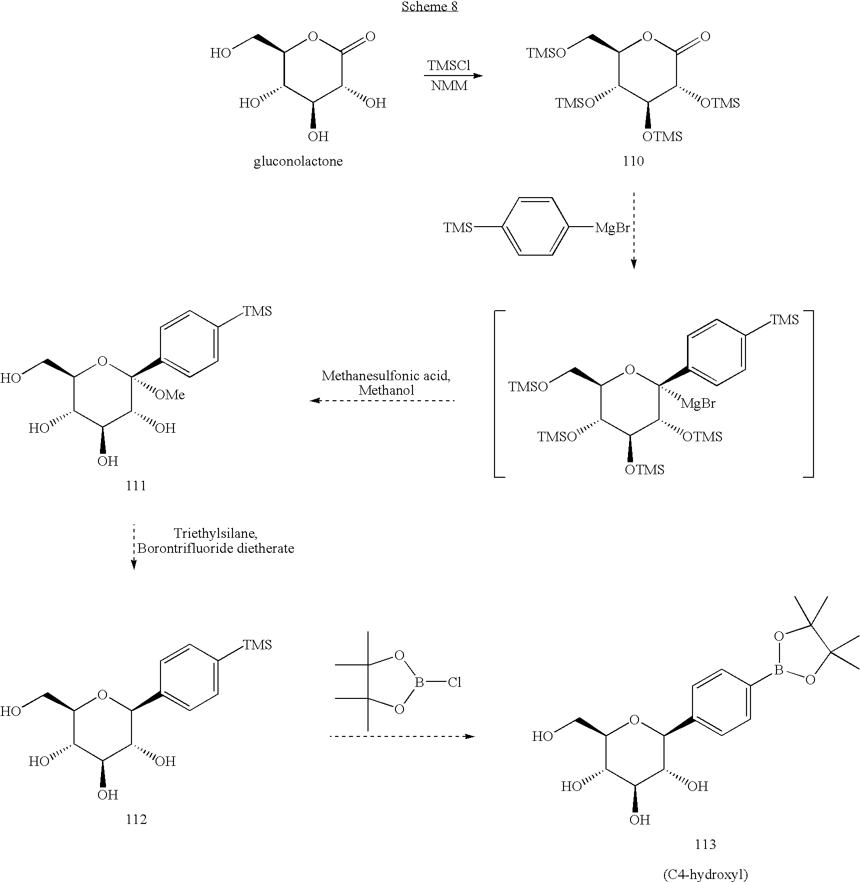

Compound 111 can be converted to C4-acetyl by slight variations as shown below in Scheme 9

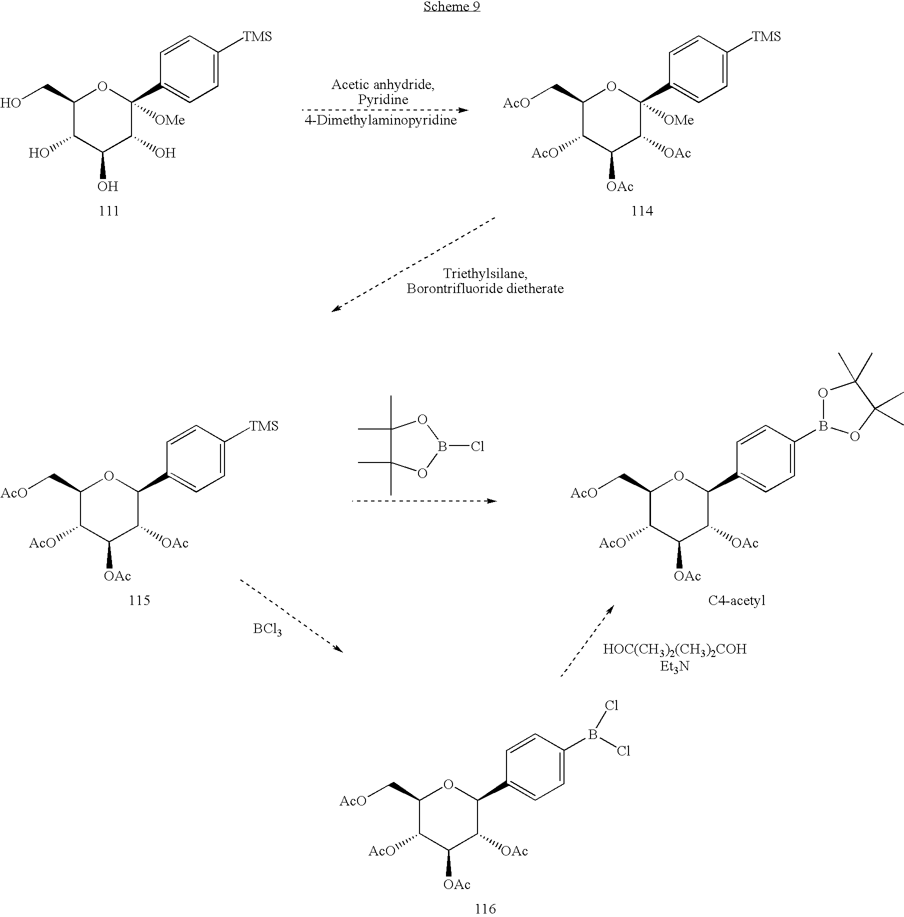

1. A process for preparing a compound of structure 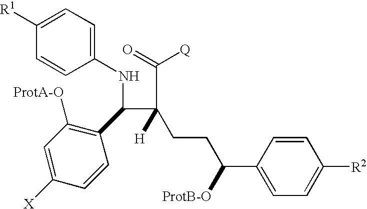
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, an allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
Q is a chiral auxiliary, said chiral auxiliary chosen from single enantiomers of triphenyl glycol and cyclic and branched nitrogen-containing moieties possessing at least one chiral center,
said process comprising reacting a compound of formula 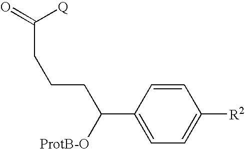
with a compound of formula

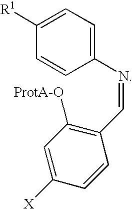

2. A process according to claim 1 for preparing a compound of structure 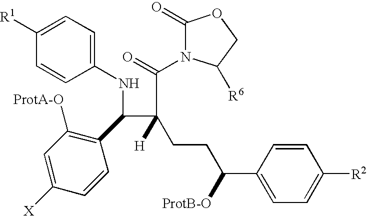
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁶ is phenyl or benzyl;
said process comprising reacting a compound of formula 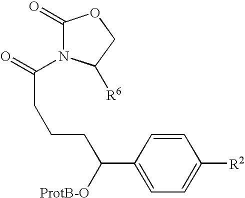
with a compound of formula

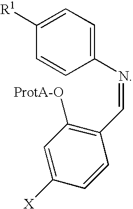

3. A process according to claim 2 comprising reacting a compound of formula 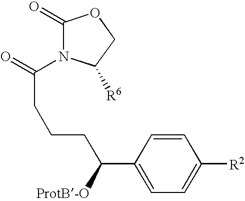
wherein
ProtB′-O— is a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester,
with a Lewis acid and a compound of formula

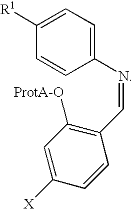

4. A process according to claim 2 comprising the sequential steps of
a. reacting a compound of formula 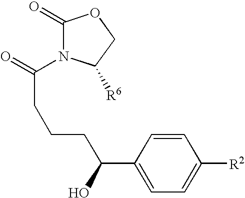
with a trialkylhalosilane in the presence of a base, followed by
b. a Lewis acid, followed by
c. a compound of formula

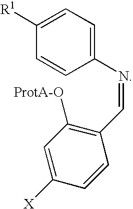

5. A process according to claim 2 wherein R¹ and R² are chosen from H and halogen; and
ProtA-O— is chosen from methoxymethyl ether, allyl ether, t-butyl ether, benzyl ether, trimethylsilyl ether, t-butyldimethylsilyl ether and t-butyldiphenylsilyl ether;

6. A process according to claim 2 wherein said compound of formula 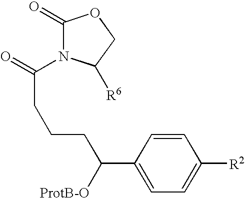
is reacted with said compound
of formula 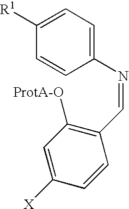
in the presence of a Lewis acid, wherein said Lewis acid is a halide of a Group 3, 4, 13 or 14 metal.

7. A process according to claim 6 wherein said Lewis acid is titanium tetrachloride.

8. A process according to claim 4 wherein
R¹ is hydrogen;
R² is fluorine;
X is bromine; and
ProtA-O— is benzyl ether.

9. A process according to claim 2 comprising
a. reacting a compound of formula 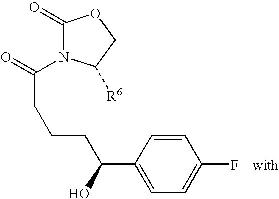
trimethylchlorosilane in the presence of a tertiary amine to provide a silyl-protected benzyl alcohol; and
b. reacting said silyl-protected benzyl alcohol with titanium tetrachloride and an imine of formula 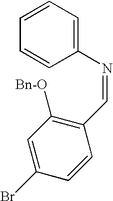
to provide a compound of formula

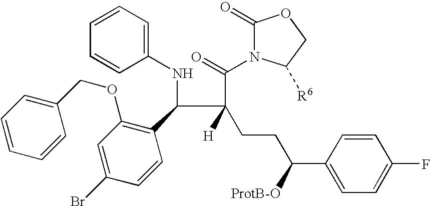

10. A process for preparing a compound of structure 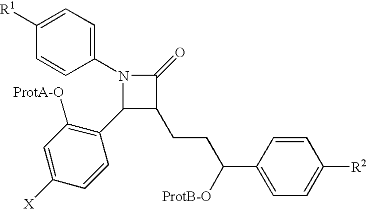
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester;
said process comprising cyclizing a compound of formula 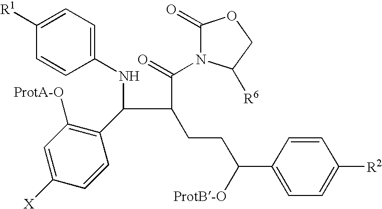
wherein
R⁶ is phenyl or benzyl; and
ProtB′-O— is a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester.

11. A process according to claim 10 comprising reacting a compound of formula 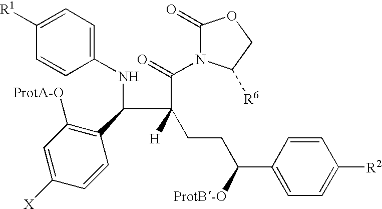
with N,O-bistrimethylsilylacetamide and a source of fluoride ion.

12. A process according to claim 11 wherein said source of fluoride ion is tetrabutylammonium fluoride.

13. A process according to claim 12 wherein
R¹ is hydrogen;
R² is fluorine;
X is bromine;
ProtA is benzyl; and
ProtB′ is silyl.

14. A process according to claim 13 wherein ProtB′ is chosen from t-butyldimethylsilyl and trimethylsilyl.

15. A process for preparing a 4-biphenylylylazetidinone of formula 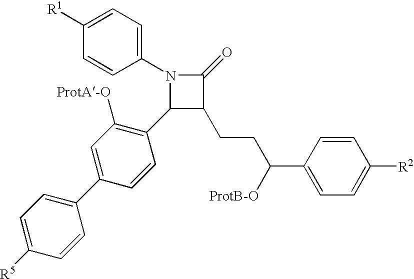
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁵ is a sugar or protected sugar;
said process comprising reacting a 4-phenylazetidin-2-one of formula 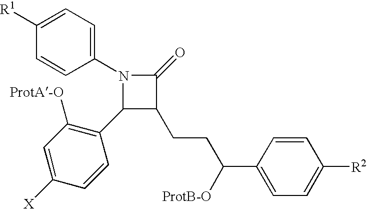
wherein
X is chosen from iodine, bromine, chlorine, toluenesulfonyl, methanesulfonyl and trifluoromethanesulfonyl;
with a phenyl component of formula 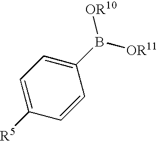
wherein
R¹⁰ and R¹¹ are independently selected from H and (C₁-C₆) alkyl, or R¹⁰ and R¹¹ together form a 5-6 membered ring.

16. A process for preparing a 4-biphenylylazetidinone of formula 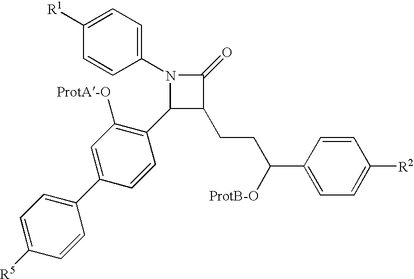
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁵ is a sugar or protected sugar;
said process comprising reacting a 4-phenylazetidin-2-one of formula 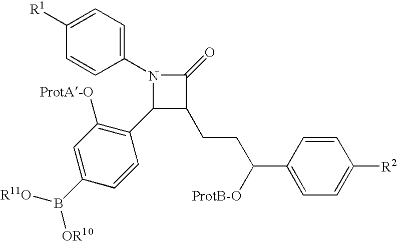
wherein
R¹⁰ and R¹¹ are independently selected from H and (C₁-C₆) alkyl, or R¹⁰ and R¹¹ together form a 5-6 membered ring;
with a phenyl component of formula 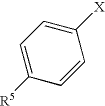
wherein
X is chosen from iodine, bromine, chlorine, toluenesulfonyl, methanesulfonyl and trifluoromethanesulfonyl.

17. A process according to claim 15 wherein said reacting a 4-phenylazetidin-2-one with a phenyl component is carried out with a phosphine, a palladium salt and a base.

18. A process according to claim 15 comprising reacting a 4-phenylazetidin-2-one of formula 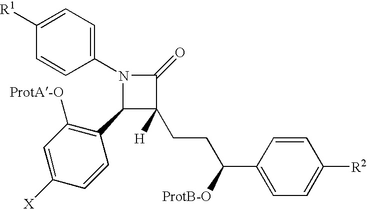
wherein
ProtA′-O— is chosen from methoxymethyl ether, t-butyl ether, silyl ether, and benzyl ether; and
ProtB-O— is chosen from HO— and silyl ether; with 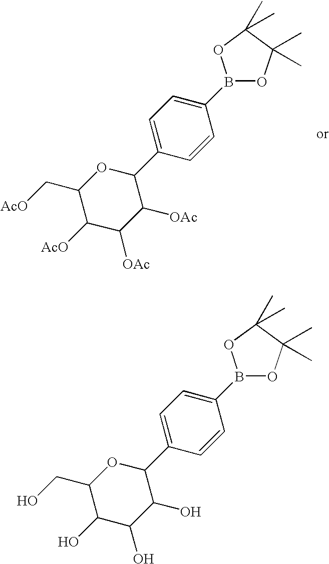
in the presence of a phosphine, a palladium salt and a base.

19. A process according to claim 16 comprising reacting a 4-phenylazetidin-2-one of formula 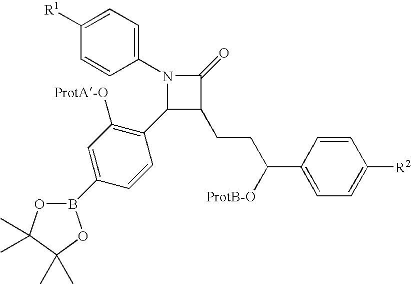
wherein
ProtA′-O— is chosen from methoxymethyl ether, t-butyl ether, silyl ether, and benzyl ether; and
ProtB-O— is chosen from HO— and silyl ether; with 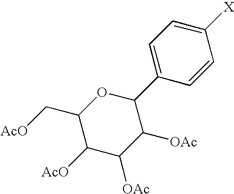
in the presence of a phosphine, a palladium salt and a base.

20. (canceled)

21. A process according to claim 15 wherein R¹ is hydrogen and R² is fluorine.

22. A process for preparing a compound of formula XII: 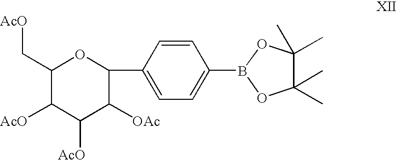
comprising reacting XV 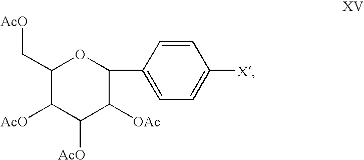
wherein X′ is Br or Cl, with bis(pinacolato)diboron in the presence of a palladium catalyst, a trivalent phosphine and a base to produce XII.

23. A process according to claim 22 wherein X′ is Cl, said palladium catalyst is bis(dibenzylideneacetone) palladium [(dba)₂Pd]; said phosphine is tricyclohexyl phosphine; said base is potassium acetate; and the reaction is carried out in diglyme at 150-175° C.

24. A process for preparing a compound of formula XIII 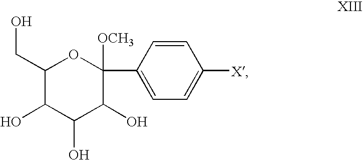
wherein X′ is Br or Cl, comprising reacting a silylated sugar lactone of formula 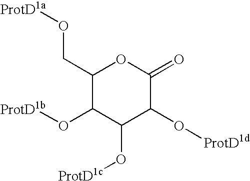
wherein ProtD^{1a}, ProtD^{1b}, ProtD^{1c} and ProtD^{1d} are trialkylsilyl groups, with a Grignard reagent followed by methanolysis.

25. A process according to claim 24 wherein said Grignard reagent is 4-chlorophenylmagnesium bromide and methanolysis is accomplished with methanesulfonic acid in methanol.

26. A process for preparing a compound of formula XII: 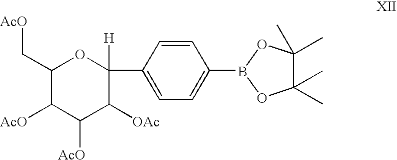
comprising:
(1) treating a protected sugar lactone of formula 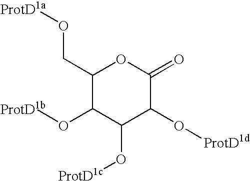
wherein ProtD^{1a}, ProtD^{1b}, ProtD^{1c} and ProtD^{1d} are trialkylsilyl groups, with a Grignard reagent
followed by methanol and an acid to provide a compound of formula XIII: 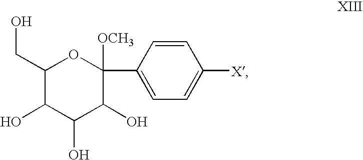
wherein X′ is Br or Cl;
(2) treating XIII with an excess of an acetylating reagent chosen from acetic anhydride in the presence of a base, acetyl chloride in the presence of a base, pentafluorophenyl acetate in the presence of a base and acetylimidazole in the presence of a platinum catalyst to provide XIV: 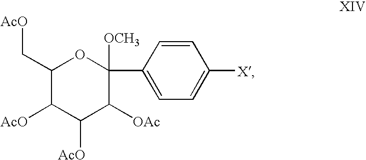
(3) reducing XIV with a silane and a Lewis acid to provide XV: 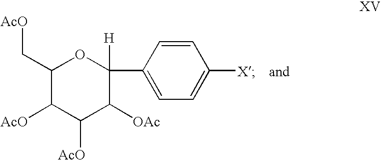
(4) reacting XV with bis(pinacolato)diboron in the presence of a palladium catalyst to produce XII.

27. A process according to claim 26 for preparing a compound of formula C4-acetyl: 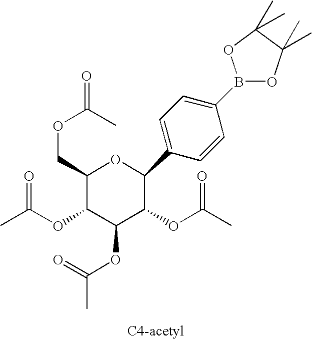
comprising:
(1) treating a protected sugar lactone of formula CC0 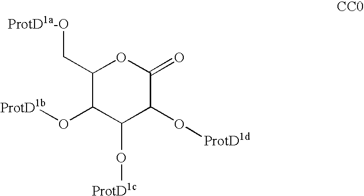
wherein ProtD^{1a}, ProtD^{1b}, ProtD^{1c} and ProtD^{1d} are trimethylsilyl or t-butylimethylsilyl groups with a Grignard reagent of formula 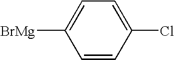
followed by methanol and an acid to provide a compound of formula CC3: 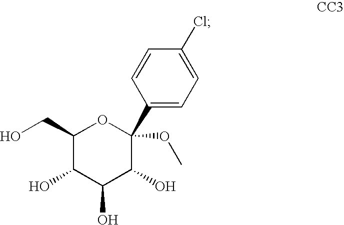
(2) treating CC3 with an excess of acetic anhydride in the presence of a base to provide CC4: 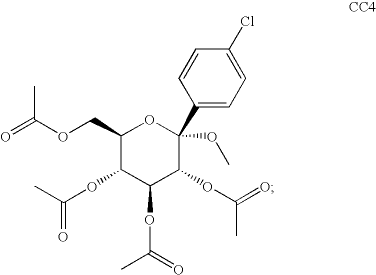
(3) reducing CC4 with triethylsilane and a Lewis acid to provide CC5: 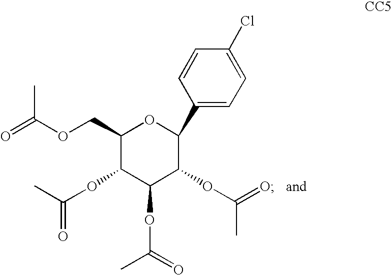
(4) reacting CC5 with bis(pinacolato)diboron in the presence of a palladium catalyst, a trivalent phosphine ligand and a base to produce C4-acetyl:

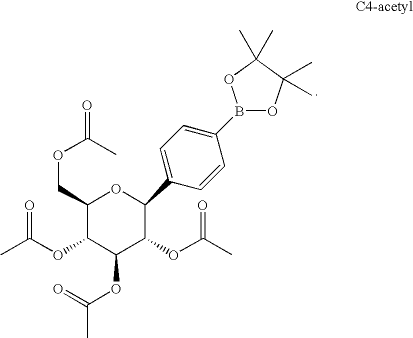

28. A process for preparing a compound of formula 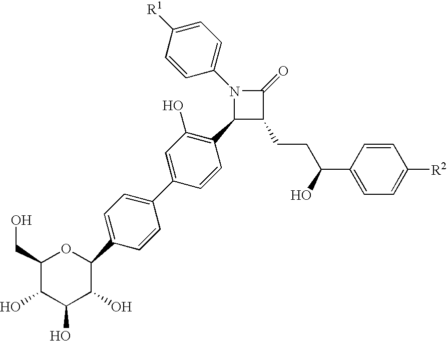
comprising reacting an azetidinone of formula 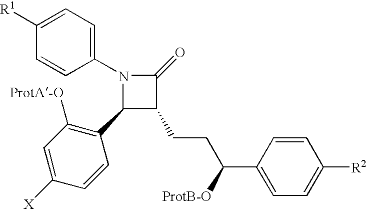
with a dioxaborole of formula 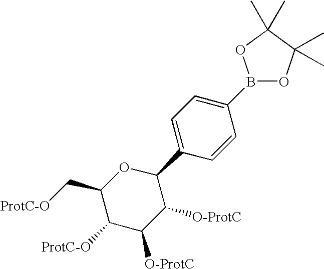
and deprotecting,
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA′-O— is a protecting group for a phenol chosen from an oxymethyl ether, a tertiary alkyl ether, a benzyl ether and a silyl ether;
ProtB-O— is —OH or silyl ether; and
ProtC-O— is a protecting group for a sugar alcohol chosen from a benzyl ether, a silyl ether and an ester.

29. A process according to claim 28 for preparing 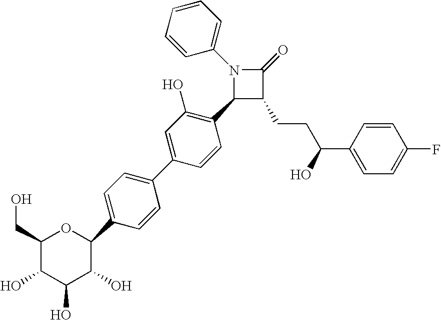
comprising reacting an azetidinone of formula 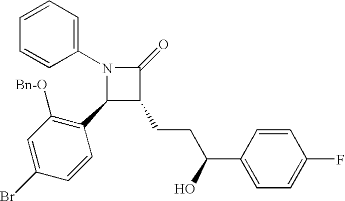
with a dioxaborole of formula 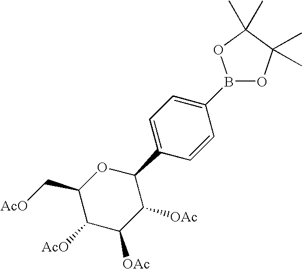
and deprotecting.

30. A process according to claim 28 wherein said azetidinone is reacted with said dioxaborole in the presence of a phosphine, a palladium salt and an alkali metal carbonate;
ProtC is acetyl and said deprotection is accomplished by hydrolysis with aqueous base; and
ProtA′ is benzyl and said deprotection is accomplished by catalytic hydrogenolysis.

31. A process according to claim 28 wherein said azetidinone is reacted with said dioxaborole in the presence of a phosphine, a palladium salt and an alkali metal carbonate;
ProtC is acetyl and said deprotection is accomplished by methanolysis in the presence of fluoride ion; and
ProtA′ is benzyl and said deprotection is accomplished by catalytic hydrogenolysis.

32. A process according to claim 28 wherein said azetidinone is obtained by cyclizing a β-aminoacyloxazolinone of formula 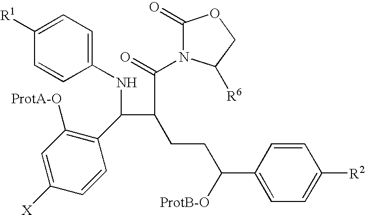
wherein
R⁶ is phenyl or benzyl.

33. A process according to claim 32 wherein said β-aminoacyloxazolinone is obtained by
reacting a compound of formula 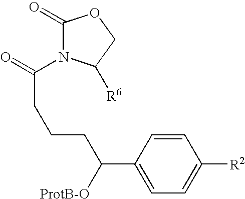
with a compound of formula

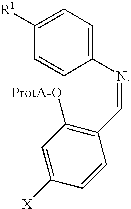

34. A process for preparing an imine of formula 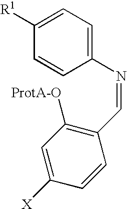
wherein
R¹ is chosen from H, halogen, —OH, and methoxy;
X is chosen from iodine, bromine, chlorine, toluenesulfonyl, methanesulfonyl and trifluoromethanesulfonyl; and
ProtA-O— is a protecting group for a phenol chosen from an oxymethyl ether, allyl ether, a tertiary alkyl ether, a benzyl ether and a silyl ether,
said process comprising reacting a phenol of formula 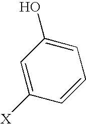
with a source of formaldehyde followed by Schiff base formation by reacting with an aniline of formula 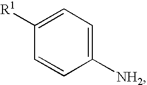
followed by protecting with ProtA.

35. A process according to claim 34 wherein ProtA is benzyl, X is bromine and R¹ is hydrogen.

36. A process for producing a compound of formula E2 from a compound of formula E1 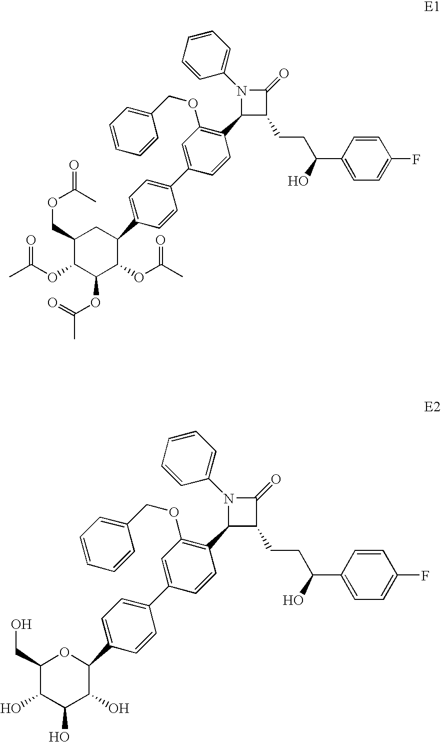
comprising treating a 0.5M solution of said compound of formula E1 in methanol with four equivalents of potassium fluoride at 35° C. to 75° C.

37. A compound of formula: 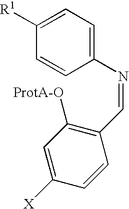
wherein
R¹ is chosen from H, halogen, —OH, and methoxy;
X is chosen from iodine, bromine, chlorine, toluenesulfonyl, methanesulfonyl and trifluoromethanesulfonyl; and
ProtA-O— is a protecting group for a phenol chosen from an oxymethyl ether, allyl ether, a tertiary alkyl ether, a benzyl ether and a silyl ether, with the proviso that when ProtA- is benzyl, R¹ is H and X is Br, the compound is solid and greater than 95% pure.

38. A compound according to claim 37 wherein R¹ is H or fluoro; X is bromine; and
ProtA-O— is a benzyl ether or silyl ether.

39. A compound of formula 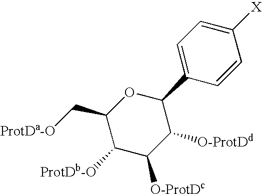
wherein
X is chosen from iodine, bromine, chlorine, toluenesulfonyl, methanesulfonyl and trifluoromethanesulfonyl; and
ProtD^{a}, ProtD^{b}, ProtD^{c} and ProtD^{d} are hydrogen or protecting groups for a sugar chosen independently from benzyl, silyl, acyl, ketal, acetal, methoxymethyl, 2-(trimethylsilyl)ethoxymethyl, allyl, 2-methylallyl and t-butyl.

40. A compound according to claim 39 wherein X is chlorine and ProtD^{a}, ProtD^{b}, ProtD^{c} and ProtD^{d} are trialkylsilyl protecting groups.

41. A compound according to claim 39 wherein X is chlorine and ProtD^{a}, ProtD^{b}, ProtD^{c} and ProtD^{d} are acetyl.

42. A compound of formula 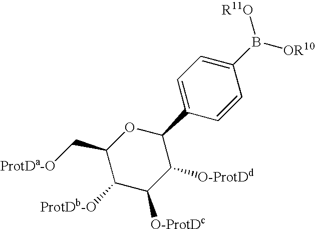
wherein
R¹⁰ and R¹¹ are independently selected from H and (C₁-C₆) alkyl, or R¹⁰ and R¹¹ together form a 5-6 membered ring; and
ProtD^{a}, ProtD^{b}, ProtD^{c} and ProtD^{d} are hydrogen or protecting groups for a sugar chosen independently from benzyl, silyl, acyl, ketal and acetal.

43. A compound according to claim 42 of formula 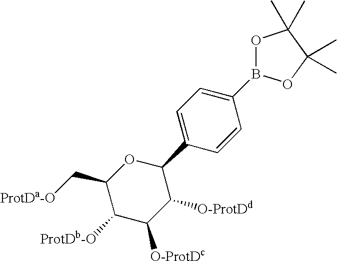
wherein
ProtD^{a}, ProtD^{b}, ProtD^{c} and ProtD^{d} are H, benzyl, silyl or acyl.

44. A compound according to claim 39 wherein ProtD^{a}, ProtD^{b}, ProtD^{c} and ProtD^{d} are all acetyl, trimethylsilyl or t-butyldimethylsilyl.

45. A compound of formula 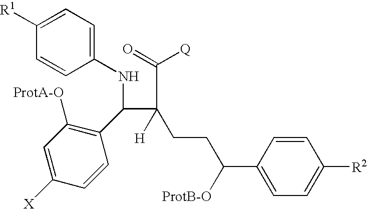
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, an allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
Q is a chiral auxiliary attached at nitrogen, said chiral auxiliary chosen from single enantiomers of cyclic and branched nitrogen-containing moieties possessing at least one chiral center.

46. A compound according to claim 45 of formula 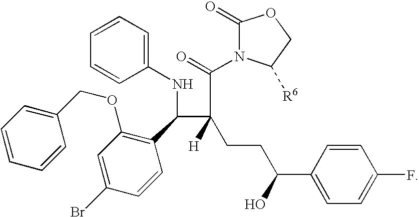
wherein R⁶ is phenyl or benzyl.

47. A compound of formula 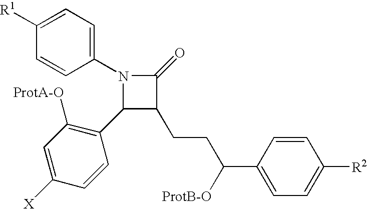
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester.

48. A compound according to claim 47 of formula

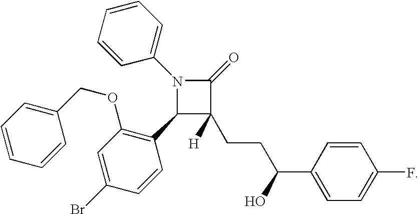

49. A compound of formula 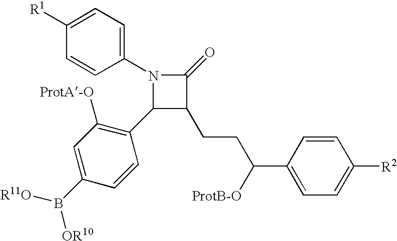
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R¹⁰ and R¹¹ are independently selected from H and (C₁-C₆) alkyl, or R¹⁰ and R¹¹ together form a 5-6 membered ring;

50. A compound according to claim 49 of formula

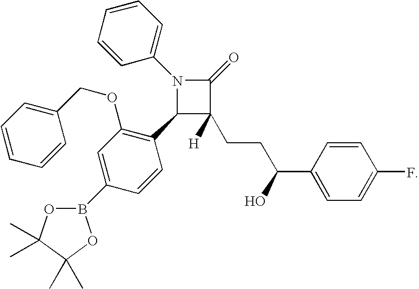

51. A compound of formula 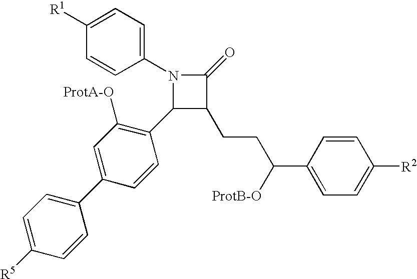
wherein
R¹ and R² are chosen from H, halogen, —OH, and methoxy;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁵ is a protected sugar.

52. A compound according to claim 51 of formula 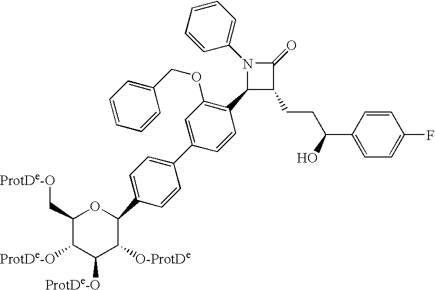
wherein
ProtD^{e} is hydrogen or acetyl.

53. A compound according to claim 52 of formula

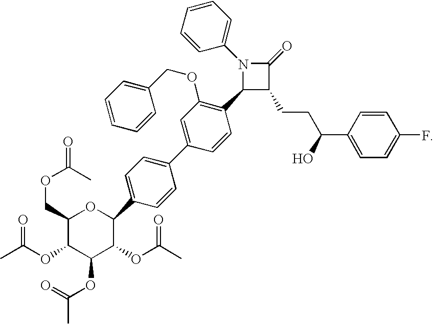

54. A compound of formula 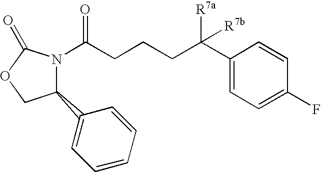
wherein one of R^{7a} and R^{7b} is H and the other is OH or taken together R^{7a} and R^{7b} are ═O.

55. A compound of formula

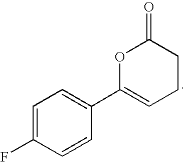

56. A process according to claim 16 wherein said reacting a 4-phenylazetidin-2-one with a phenyl component is carried out with a phosphine, a palladium salt and a base.

57. A compound according claim 42 wherein ProtD^{a}, ProtD^{b}, ProtD^{c} and ProtD^{d} are all acetyl, trimethylsilyl or t-butyldimethylsilyl.

58. A process according to claim 16 wherein R¹ is hydrogen and R² is fluorine.
